(19) United States
(12) Patent Application Publication
(10) Pub. No.: US 2005/0256582 A1
(43) Pub. Date: Nov. 17, 2005

(54) Spinal implants, including devices that reduce pressure on the annulus fibrosis

(76) Inventor: Bret A. Ferree, Cincinnati, OH (US)

Correspondence Address: John G. Posa;Gifford, Krass, Groh, Sprinkle,, Anderson & Citkowski, P.C., PO Box 7021, Troy, MI, 48007, US

(21) Appl. No.: 11/187,250

(22) Filed: Jul. 22, 2005

Related U.S. Application Data
(63) Continuation-in-part of application No. 10/120,763, filed on Apr. 11, 2002.
Application No. 10/120,763 is a continuation-in-part of application No. 09/807,820, filed on Apr. 19, 2001, now abandoned.
(63) Continuation-in-part of application No. 10/185,284, filed on Jun. 26, 2002.
Application No. 10/185,284 is a continuation-in-part of application No. 10/120,763, filed on Apr. 11, 2002.
Application No. 10/185,284 is a continuation-in-part of application No. 09/807,820, filed on Apr. 19, 2001, now abandoned.
Application No. 10/185,284 is a continuation-in-part of application No. 09/415,382, filed on Oct. 8, 1999, now Pat. No. 6,419,704.
(63) Continuation-in-part of application No. 10/303,385, filed on Nov. 25, 2002.
Application No. 10/303,385 is a continuation-in-part of application No. 09/415,382, filed on Oct. 8, 1999, now Pat. No. 6,419,704.
Application No. 10/303,385 is a continuation-in-part of application No. 10/191,639, filed on Jul. 9, 2002.
(63) Continuation-in-part of application No. 10/991,733, filed on Nov. 18, 2004.
Application No. 10/991,733 is a continuation-in-part of application No. 10/421,434, filed on Apr. 23, 2003, now Pat. No. 6,878,167.
(60) Provisional application No. 60/590,942, filed on Jul. 23, 2004.
(60) Provisional application No. 60/375,185, filed on Apr. 24, 2002.
(60) Provisional application No. 60/378,132, filed on May 15, 2002.

(51) Int. Cl. A61F 2/44

(52) U.S. Cl. 623/17.16; 623/17.11

(57) ABSTRACT

The invention broadly facilitates reconstruction of the Annulus Fibrosus (AF) or the AF and the Nucleus Pulposus (NP). Such Reconstruction prevents recurrent herniation following Microlumbar Discectomy (MLD) other procedures. The invention may also be used in the treatment of herniated discs, annular tears of the disc, or disc degeneration, while enabling surgeons to preserve the contained NP. The methods and apparatus may be used to treat discs throughout the spine including the cervical, thoracic, and lumbar spines of humans and animals. In the preferred embodiment, a spinal repair system according to the invention comprises a first end portion adapted for placement within an intervertebral body, a second end portion adapted for placement within an adjacent intervertebral body, and a bridge portion connecting the first and second end portions, the bridge portion being adapted to span a portion of an intervertebral disc space and prevent excessive outward bulging.

### REFERENCE TO RELATED APPLICATIONS

This application claims priority from U.S. Provisional Patent Application Ser. No. 60/590,942, filed Jul. 23, 2004.

This application is also a continuation-in-part of U.S. patent application Ser. No. 10/120,763, filed Apr. 11, 2002, which is a continuation-in-part of U.S. patent application Ser. Nos. 09/807,820, filed Apr. 19, 2001, now abandoned, which is a U.S. national phase application of PCT/US00/14708, filed May 30, 2000; and 09/638,241, filed Aug. 14, 2000; and 09/454,908, filed Dec. 3, 1999, now U.S. Pat. No. 6,491,724; and 09/639,309, filed Aug. 14, 2000, now U.S. Pat. No. 6,419,702; and 09/690,536, filed Oct. 16, 2000, now U.S. Pat. No. 6,371,990, which is a continuation-in-part of U.S. patent application Ser. Nos. 09/638,726, filed Aug. 14, 2000, now U.S. Pat. No. 6,340,369; and 09/415,382, filed Oct. 8, 1999, now U.S. Pat. No. 6,419,704.

This application is also a continuation-in-part of U.S. patent application Ser. No. 10/185,284, filed Jun. 26, 2002, which is a continuation-in-part of U.S. patent application Ser. Nos. 10/120,763, filed Apr. 11, 2002; 09/807,820, filed Apr. 19, 2001, now abandoned; and 09/415,382, filed Oct. 8, 1999, now U.S. Pat. No. 6,419,704, and 10/191,639, filed Jul. 9, 2002.

This application is also a continuation-in-part of U.S. patent application Ser. No. 10/303,385, filed Nov. 25, 2002, which is a continuation-in-part of U.S. patent application Ser. No. 09/415,382, filed Oct. 8, 1999, now U.S. Pat. No.

This application is also a continuation-in-part of U.S. patent application Ser. No. 10/991,733, filed Nov. 18, 2004, which is a continuation-in-part of U.S. patent application Ser. No. 10/421,434, filed Apr. 23, 2003, now U.S. Pat. No. 6,878,167, which claims priority from U.S. Provisional Patent Application Ser. Nos. 60/375,185, filed Apr. 24, 2002 and 60/378,132, filed May 15, 2002. The entire content of each application and patent is incorporated herein by reference.

### BACKGROUND OF THE INVENTION

The human intervertebral disc is an oval to kidney bean shaped structure of variable size depending on the location in the spine. The outer portion of the disc is known as the annulus fibrosis (AF). The AF is formed of 10 to 60 fibrous bands. The fibers in the bands alternate their direction of orientation by 30 degrees between each band. The orientation serves to control vertebral motion (one half of the bands tighten to check motion when the vertebra above or below the disc are turned in either direction).

The AF contains the nucleus. The nucleus pulpous serves to transmit and dampen axial loads. A high water content (70-80 percent) assists the nucleus in this function. The water content has a diurnal variation. The nucleus imbibes water while a person lies recumbent. Activity squeezes fluid from the disc. Nuclear material removed from the body and placed into water will imbibe water swelling to several times its normal size. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per micro liter.

The disc changes with aging. As a person ages the water content of the disc falls from approximately 85 percent at birth to 70 percent in the elderly. The ratio of chondroitin sulfate to keratin sulfate decreases with age. The ratio of chondroitin 6 sulfate to chondroitin 4 sulfate increases with age. The distinction between the annulus and the nucleus decreases with age. These changes are known as disc degeneration. Generally disc degeneration is painless.

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component. Although the use of resilient materials has been proposed, the need remains for further improvements in the way in which prosthetic components are incorporated into the disc space, and in materials to ensure strength and longevity. Such improvements are necessary, since the prosthesis may be subjected to 100,000,000 compression cycles over the life of the implant.

Current nucleus replacements (NRs) may cause low back pain, if too much pressure is applied to the AF. As discussed in my co-pending U.S. patent application Ser. No. 10/407,554 and U.S. Pat. No. 6,878,167, the content of each being incorporated herein by reference, the posterior portion of the AF has abundant pain fibers.

Herniated Nucleus Pulposus (HNP) occurs from tears in the AF. The herniated NP often allies pressure on the nerves or spinal cord. Compressed nerves cause back and leg or arm pain. Although a patient's symptoms result primarily from pressure by the NP, the primary pathology lies in the AF.

Surgery for HNP, Microlumbar Discectomy (MLD) addresses only the NP. The opening in the AF is enlarged during surgery, further weakening the AF. Surgeons also remove generous amounts of the NP to reduce the risk of extruding additional pieces of NP through the defect in the AF. Although MLD decreases or eliminates a patient's leg or arm pain, the procedure damages weakened discs.

### SUMMARY OF THE INVENTION

The invention broadly facilitates reconstruction of the Annulus Fibrosus (AF) or the AF and the Nucleus Pulposus (NP). Such Reconstruction prevents recurrent herniation following Microlumbar Discectomy (MLD). The invention may also be used in the treatment of herniated discs, annular tears of the disc, or disc degeneration, while enabling surgeons to preserve the contained NP. The methods and apparatus may be used to treat discs throughout the spine including the cervical, thoracic, and lumbar spines of humans and animals.

The invention also enables surgeons to reconstruct the AF and replace or augment the NP. Novel Nucleus Replacements (NR) may be added to the disc. Annulus Reconstruction prevents extrusion of the NRs through holes in the AF. The NRs and the AF reconstruction prevent excessive pressure on the AF. Excessive pressure on the AF may cause back or leg pain. The NRs may be made of natural or synthetic materials. Synthetic NRs are preferably made of polymers including polyurethane, silicon, hydrogel, or other elastomers.

In the preferred embodiment, a spinal repair system according to the invention comprises a first end portion adapted for placement within an intervertebral body, a second end portion adapted for placement within an adjacent intervertebral body, and a bridge portion connecting the first and second end portions, the bridge portion being adapted to span a portion of an intervertebral disc space and prevent excessive outward bulging.

The first and second end portions may be composed of a rigid biocompatible material, including metals, alloys, or ceramics, and the bridge portion is composed of a flexible, braided or mesh material. Preferably, however, the first and second end portions are composed of allograft bone and the bridge portion is composed of allograft annulus fibrosis. A single piece of allograft tissue, such as fascia, may alternatively be used. The system may further include screws and/or plates to hold the first and second end portions into respective vertebral bodies.

In one configuration the first and second end portions are elongate, and the bridge portion spans the end portions in a plane parallel to the end portions. The system may further comprise slotted bone dowels into which the end portions are received, and the bridge portion extends through one slot and into the other when implanted. The system may further include an artificial disc replacement (ADR) defining a volume, with the bridge portion extending through at least a portion of the volume of the ADR.

The end and bridge portions may together form a cylindrical shape. At least one of the end portions may be threaded. One or both of the end portions may be configured for bony ingrowth. Various instruments and methods are also disclosed.

A spinal repair method according to the invention includes the steps of forming a first hole or channel in a first intervertebral body, placing the first end portion into the first hole or channel, forming a second hole or channel in an adjacent intervertebral body, and placing the second end portion into the second hole or channel such that the bridge portion spans a hole or defect in an annulus fibrosis. The end portions may then be secured with screws. The step of providing the system of claim 1 may include harvesting the portions from a human or animal donor, with the end portions comprising intervertebral bone and the bridge portion comprises annulus fibrosis still attached to the end portions.

### BRIEF DESCRIPTION OF THE DRAWINGS

FIG. 1A is a lateral view of a “curtain” annulus augmentation device;

FIG. 1B is an anterior view of the embodiment of the invention drawn in FIG. 1A;

FIG. 1C is a lateral view of the spine and the embodiment of the invention drawn in FIG. 1A;

FIG. 1D is an axial cross section of the disc and device drawn in FIG. 1C;

FIG. 2A is an anterior view of the spine and an alternative embodiment with the disc not included to better illustrate the device;

FIG. 2B is a lateral view of the embodiment of the device drawn in FIG. 2A;

FIG. 2C is an anterior view of a segment of a cadaver spine;

FIG. 2D is an exploded anterior view of the cadaver spine drawn in FIG. 2C;

FIG. 2E is an axial cross section of a cadaver disc;

FIG. 2F is an axial cross section of a cadaver disc;

FIG. 3A is an anterior view of a novel ADR that may be placed anterior to the device drawn in FIG. 1C;

FIG. 3B is a lateral view of the ADR drawn in FIG. 3A;

FIG. 4A is a lateral view of the spine used to illustrate methods to insert intradiscal devices;

FIG. 4B is a lateral view of the spine drawn in FIG. 4A;

FIG. 4C is a lateral view of the spine, the embodiment of the device drawn in FIG. 2B;

FIG. 4D is a lateral view of the spine;

FIG. 4E is a lateral view of the spine an alternative embodiment of the plates drawn in FIG. 4D;

FIG. 5A is an exploded lateral view of an alternative embodiment of the present invention;

FIG. 5B is an exploded lateral view of the embodiment of the device drawn in FIG. 5A;

FIG. 5C is a lateral view of the assembled device drawn in FIG. 5B;

FIG. 5D is an oblique view of the screw drawn in FIG. 5A;

FIG. 5E is an anterior view of the assembled device drawn in FIG. 5C;

FIG. 5F is a lateral view of the spine and the embodiment of the device drawn in FIG. 5E;

FIG. 6 is lateral view of the device drawn in FIG. 1A incorporated into the posterior aspect of the NR;

FIG. 7A is a lateral view of the spine;

FIG. 7B is a lateral view of a guide that fits over the guide wire drawn in FIG. 7A;

FIG. 7C is a lateral view of the spine and the guide drawn in FIG. 7B;

FIG. 8A is a lateral view of an alternative embodiment of the NR drawn in FIG. 6, wherein the embodiment of the invention drawn in FIG. 1A is incorporated into the body of a NR;

FIG. 8B is an anterior view of the embodiment of the NR drawn in FIG. 8A;

FIG. 8C is sagittal cross section of the embodiment of the NR drawn in FIG. 8A;

FIG. 8D is an axial cross section of the embodiment of the NR drawn in FIG. 8A;

FIG. 8E is a coronal cross section of the embodiment of the NR drawn in FIG. 8A;

FIG. 8F is a lateral view of the spine and the embodiment of the NR drawn in FIG. 8A;

FIG. 9A is an anterior view of an alternative embodiment of the present invention;

FIG. 9B is an anterior view of the spine and the embodiment of the NR drawn in FIG. 9A;

FIG. 9C is a lateral view of an embodiment similar to that drawn in FIG. 9A, wherein the retention members have holes to accept tools to insert the NR;

FIG. 9D is an anterior view of the embodiment of the NR drawn in FIG. 9A;

FIG. 9E is an anterior view of the L5 vertebra, the sacrum, and an alternative embodiment of an NR with a retention member;

FIG. 9F is an anterior view of the spine drawn in FIG. 9E. Plates are shown holding the NR and the vertebral fragment on the spine.

FIG. 9G is an axial cross section of the embodiment of the NR drawn in FIG. 9E;

FIG. 9H is a lateral view of the L5 vertebra, the sacrum, and the embodiment of the NR drawn in FIG. 9E;

FIG. 10A is a lateral view of a version of the NR drawn in FIG. 8A that incorporates a balloon on the posterior aspect of the device;

FIG. 10B is a lateral view of the spine and the embodiment of the NR drawn in FIG. 8A;

FIG. 11A is an axial cross section an alternative embodiment of the invention wherein a flexible retention component lies between separate anterior and posterior cushion components;

FIG. 11B is an exploded axial cross section of the embodiment of the invention drawn in FIG. 11A prior to surrounding the components with an elastic material;

FIG. 11C is a sagittal cross section of the embodiment of the NR drawn in FIG. 11A;

FIG. 11D is a lateral view of the embodiment of the NR drawn in FIG. 11A;

FIG. 11E is a view of the top of the embodiment of the NR drawn in FIG. 11A;

FIG. 11F is a lateral view of the spine and the embodiment of the NR drawn in FIG. 11A;

FIG. 12A is an axial cross section of an alternative embodiment of the present invention;

FIG. 12B is a lateral view of the embodiment of the NR drawn in FIG. 12A;

FIG. 12C is a view of the top of the embodiment of the device drawn in FIG. 12B;

FIG. 12D is a lateral view of the L5 vertebra, the sacrum, and the embodiment of the NR drawn in FIG. 12A;

FIG. 13A is an oblique view of an alternative embodiment of the invention drawn in FIG. 2B;

FIG. 13B is an oblique view of an alternative embodiment of the invention drawn in FIG. 13A. The bone ends have at least one flat surface along their sides.

FIG. 13C is an oblique view of an alternative embodiment of the invention drawn in FIG. 13B;

FIG. 13D is an oblique view of an alternative embodiment of the invention drawn in FIG. 13C;

FIG. 14A is an oblique view of an alternative embodiment of the invention drawn in FIG. 13A;

FIG. 14B is an oblique view of the embodiment of the invention drawn in FIG. 14A;

FIG. 15 is an oblique view of an alternative embodiment of the invention drawn in FIG. 13A;

FIG. 16 is an alternative embodiment of the invention drawn in FIG. 15;

FIG. 17 is an alternative embodiment of the invention drawn in FIG. 16;

FIG. 18 is an alternative embodiment of the invention drawn in FIG. 13A.;

FIG. 19 is an alternative embodiment of the invention drawn in FIG. 19;

FIG. 20 is a lateral view of the spine;

FIG. 21A is a lateral view of a portion of the lumbar spine;

FIG. 21B is an exploded lateral view of the spine drawn in FIG. 21A;

FIG. 21C is a lateral view of one disc unit drawn in FIG. 21B;

FIG. 21D is an axial cross section through the disc drawn in FIG. 21C;

FIG. 21E is an exploded lateral view of the embodiment of the invention drawn in FIG. 21C;

FIG. 21F is an exploded axial view of the embodiment of the invention drawn in FIG. 21D;

FIG. 22A is an axial cross section of a disc donor unit (disc with portions of the vertebra above and below the disc);

FIG. 22B is an exploded axial cross section of the donor disc unit drawn in FIG. 22A;

FIG. 23A is an oblique view of an alternative embodiment of the invention drawn in FIG. 13A;

FIG. 23B is a lateral view of a donor disc unit;

FIG. 24A is a lateral view of the embodiment of the invention drawn in FIG. 13A;

FIG. 24B is a lateral view of the embodiment of the invention drawn in FIG. 24A;

FIG. 25 is a partial sagittal cross section of a portion of the lumbar spine and the embodiment of the invention drawn in FIG. 13A;

FIG. 26A is a lateral view of the spine and the first step in the method of inserting the device drawn in FIG. 13A;

FIG. 26B is a sagittal cross section of the spine and the embodiment of the invention drawn in FIG. 26A;

FIG. 26C is a sagittal cross section of the spine and the embodiment of the invention drawn in FIG. 26B;

FIG. 26D is a sagittal cross section of a portion of the spine;

FIG. 26E is a sagittal cross section of a portion of the spine and the embodiment of the invention drawn in FIG. 13A;

FIG. 26F is sagittal cross section of a portion of the spine and the embodiment of the invention drawn in FIG. 13A;

FIG. 26G is a sagittal cross section of the spine and the embodiment of the invention drawn in FIG. 26F;

FIG. 26H is a lateral view of a portion of the spine and the embodiment of the invention drawn in FIG. 26G;

FIG. 27 is a partial sagittal cross section of a portion of the spine and an alternative fixation method;

FIG. 28A is a partial sagittal cross section of a portion of the spine and the embodiment of the invention drawn in FIG. 27;

FIG. 28B is a partial sagittal cross section of a portion of the spine and an alternative embodiment of the invention drawn in FIG. 28A;

FIG. 29A is an oblique view of a novel guide;

FIG. 29B is a view of the top of a portion of the embodiment of the invention drawn in FIG. 29A;

FIG. 29C is a view of the top of a portion of an alternative embodiment of the invention drawn in FIG. 29B;

FIG. 30A is a view of the top of a portion of an alternative embodiment of the invention including a retractable component;

FIG. 30B is a view of the top of a portion of the embodiment of the invention drawn in FIG. 30A;

FIG. 31A is a partial sagittal cross section of a portion of the spine;

FIG. 31B is a lateral view of a vertebra;

FIG. 31C is a lateral view of a vertebra;

FIG. 31D is a lateral view of a vertebra;

FIG. 32 is an exploded oblique view of an alternative embodiment of the invention;

FIG. 33 is a sagittal cross section of a portion of the spine and an alternative embodiment of the invention drawn in FIG. 26H;

FIG. 34 is an axial cross section of the disc following MTD;

FIG. 35A is an axial cross section of a disc and the embodiment of the invention drawn in FIG. 33;

FIG. 35B is an axial cross section of a disc and an alternative embodiment of the invention drawn in FIG. 35A;

FIG. 36A is lateral view of novel distractor blades used to facilitate insertion of the graft drawn in FIG. 33;

FIG. 36B is a view of the cephalad side of the embodiment of the invention drawn in FIG. 36A;

FIG. 36C is lateral view of a portion of the spine and the embodiment of the invention drawn in FIG. 36B;

FIG. 36D is view of the dorsal aspect of adjacent spinuos processes and the tips of the distractor taught in FIG. 36C;

FIG. 37 is an oblique view of a novel instrument that may be used to help guide insertion of the interference screws;

FIG. 38A is a sagittal cross section of a sleeve, a screwdriver, and alternative embodiment of the cap instrument drawn in FIG. 37;

FIG. 38B is a view of the top of the cap instrument drawn in FIG. 38A;

FIG. 38C is a view of the bottom of the embodiment of the invention drawn in FIG. 38B;

FIG. 39A is an oblique view of an alternative embodiment of the invention drawn in FIG. 13A;

FIG. 39B is a sagittal cross section of a portion of the spine and the embodiment of the invention drawn in FIG. 39A;

FIG. 39C is a lateral view of the embodiment of the tool drawn in FIG. 39B;

FIG. 39D is an oblique view of the embodiments of the invention drawn in FIGS. 39A and 39C;

FIG. 40A is an oblique view of an alternative embodiment of the invention drawn in FIG. 13B;

FIG. 40B is a lateral view of the embodiments of the invention drawn in FIGS. 39C and 40A;

FIG. 41 is an oblique view of an alternative embodiment of the invention drawn in FIG. 13A;

FIG. 42A is a view of a portion of an alternative embodiment of the invention drawn in FIG. 39C;

FIG. 42B is a lateral view of the embodiments of the graft drawn in FIGS. 41 and 42A;

FIG. 43 is a lateral view of an alternative embodiment of the invention drawn in FIG. 42A;

FIG. 44 is a lateral view of an alternative embodiment of the invention drawn in FIG. 43 and a graft similar to that drawn in FIG. 13A;

FIG. 45 is an oblique view of an alternative embodiment of the invention drawn in FIG. 41;

FIG. 46 is a lateral view of alternative embodiments of the invention drawn in FIG. 39C and 40A;

FIG. 47 is a lateral view of an alternative embodiment of the invention drawn in FIG. 46;

FIG. 48 is a lateral view of embodiments of the invention drawn in FIG. 46 and 29A;

FIG. 49A is a view of a portion of the back;

FIG. 49B is a partial sagittal cross section of the back, a muscle retractor, the retractor sleeve drawn in FIG. 29A, and a drill bit;

FIG. 50A is a partial sagittal cross section of the back and an alternative embodiment of the invention drawn in FIG. 49B;

FIG. 50B is a lateral view of the flexible drill bit drawn in FIG. 50A. The shaft of the drill bit is flexible.

FIG. 51 is a sagittal cross section of an alternative embodiment of the invention drawn in FIG. 33;

FIG. 52 is a sagittal cross section of an alternative embodiment of the invention drawn in FIG. 51;

FIG. 53 is an axial cross section of a disc and an alternative embodiment of the invention drawn in FIG. 35A;

FIG. 54 is an axial cross section of a disc and an alternative embodiment of the invention drawn in FIG. 35B;

FIG. 55 is an axial cross section of a disc and an alternative embodiment of the invention drawn in FIG. 35B;

FIG. 56 is a sagittal cross section of the spine and an alternative embodiment of the invention drawn in FIG. 46;

FIG. 57 is an axial cross section of disc, a novel Nucleus Replacement (NR), and an alternative embodiment of the invention drawn in FIG. 54;

FIG. 58 is an axial cross section of a disc, an alternative embodiment of the NR drawn in FIG. 57, and an alternative embodiment of the invention drawn in FIG. 33;

FIG. 59 is an oblique view of an alternative embodiment of the invention;

FIG. 60A is a lateral view of an alternative embodiment of the invention drawn in FIG. 13A;

FIG. 60B is a lateral view of the spine and the embodiment of the invention drawn in FIG. 60A;

FIG. 61 is an axial cross section of a disc;

FIG. 62 is a sagittal cross section of the spine and an alternative embodiment of the invention drawn in FIG. 25;

FIG. 63 is a sagittal cross section of the spine and an alternative embodiment of the invention drawn in FIG. 62;

FIG. 64A is a lateral view of an alternative embodiment of the retractor drawn in FIG. 29A;

FIG. 64B is an oblique view of an alternative embodiment of the invention drawn in FIG. 13A;

FIG. 64C is a partial sagittal cross section of a portion of the spine and the embodiments of the invention drawn in FIGS. 64A and 64B;

FIG. 65 is a sagittal cross section of a portion of the spine and an alternative embodiment of the invention drawn in FIG. 25. One end to the graft is held by a screw that passes through at least a portion of the pedicle of the vertebra and the graft. Pins or other fixation devices may also be passed through the pedicle and the graft.

FIG. 66 is an anterior view of the spine and an alternative embodiment of the invention drawn in FIG. 25;

FIG. 67A is a lateral view of a portion of the spine and an alternative embodiment of the invention;

FIG. 67B is a lateral view of a portion of the spine and the embodiment of the invention drawn in FIG. 67A;

FIG. 68A is a lateral view of a portion of the spine and an alternative embodiment of the invention;

FIG. 68B is a lateral view of a portion of the spine and the embodiment of the invention drawn in FIG. 68A;

FIG. 69 is an axial cross section of a disc and the embodiment of the invention drawn in FIG. 60A;

FIG. 70A is an axial cross section of the disc and an alternative embodiment of the invention;

FIG. 70B is an axial cross section of the disc and the embodiment of the invention drawn in FIG. 70A;

FIG. 70C is an axial cross section of the disc and the embodiment of the invention drawn in FIG. 70B;

FIG. 71A is a lateral view of a portion of the spine and alternative embodiment of the invention taught in FIG. 1C;

FIG. 71B is an axial cross section of the disc and the embodiment of the invention taught in FIG. 71A;

FIG. 71C is a coronal cross section of the spine and the embodiment of the invention drawn in FIG. 71A;

FIG. 72A is a lateral view of the spine and two K-wires;

FIG. 72B is a lateral view of the spine and the embodiment of the invention drawn in FIG. 72A;

FIG. 72C is a lateral view of a guide that may be used with the embodiment of the invention drawn in FIG. 72B;

FIG. 72D is a lateral view of a portion of the spine;

FIG. 72E is an oblique view of the tissue used in the embodiment of the invention drawn in FIG. 71A;

FIG. 73A is a lateral view of an alternative embodiment of the invention drawn in FIG. 72E;

FIG. 73B is an anterior view of the embodiment of the device drawn in FIG. 73A;

FIG. 73C is lateral view of the spine and the embodiment of the invention drawn in FIG. 73B;

FIG. 74 is a lateral view of the spine and an alternative embodiment of the invention drawn in FIG. 71A;

FIG. 75A is a lateral view of an alternative embodiment of the invention drawn in FIG. 13A;

FIG. 75B is a lateral view of the spine, the embodiment of the invention drawn in FIG. 75A, and an alternative embodiment of the invention drawn in FIG. 25;

FIG. 75C is a lateral view of an alternative embodiment of the invention drawn in FIG. 75A;

FIG. 75D is an oblique view of a portion of the embodiment of the invention drawn in FIG. 75A;

FIG. 76A is a lateral view of an alternative embodiment of the invention drawn in FIG. 75A;

FIG. 76B is an axial cross section of a disc and the embodiment of the invention drawn in FIG. 76A;

FIG. 77A is an exploded lateral view of an alternative embodiment of the invention drawn in FIG. 75A;

FIG. 77B is a lateral view of the embodiment of the invention drawn in FIG. 77A;

FIG. 78A is a lateral view of an alternative embodiment of the invention dry in FIG. 77A;

FIG. 78B is a lateral view of the embodiment of the invention drawn in FIG. 78A;

FIG. 79A is an exploded lateral view of an alternative embodiment of the invention drawn in FIG. 78A;

FIG. 79B is a lateral view of the assembled embodiment of the invention drawn in FIG. 79A;

FIG. 80A is an exploded lateral view of an alternative embodiment of the invention drawn in FIG. 79A;

FIG. 80B is a lateral view of the assembled embodiment of the invention drawn in FIG. 80A;

FIG. 80C is a sagittal cross section of a portion of the embodiment of the invention drawn in FIG. 80B;

FIG. 81A is the view of the top of a portion of the embodiment of the invention drawn in FIG. 79A;

FIG. 81B is sagittal cross section of the embodiment of the invention drawn in FIG. 81A;

FIG. 81C is a sagittal cross section of the embodiment of the invention drawn in FIG. 81B;

FIG. 82A is an exploded lateral view of an alternative embodiment of the invention drawn in FIG. 80A;

FIG. 82B is a lateral view of an assembled embodiment of the invention drawn in FIG. 82A;

FIG. 83A is an axial cross section through the flexible component of an alternative embodiment of the invention drawn in FIG. 75A;

FIG. 83B is a lateral view of the embodiment of the invention drawn in FIG. 83A;

FIG. 83C is a lateral view of the embodiment of the invention drawn in FIG. 83B, an interference screw, a screwdriver, and a novel guide;

FIG. 83D is an oblique view of the tip of the guide drawn in FIG. 83C;

FIG. 84A is an exploded lateral view of an alternative embodiment of the invention drawn in FIG. 75C;

FIG. 84B is lateral view of the assembled embodiment of the invention drawn in FIG. 84A;

FIG. 85 is a sagittal cross section of a portion of the spine and an alternative embodiment of the inventions drawn in FIGS. 75C and 70C;

FIG. 86A is an axial cross section of a disc and the embodiment of the invention drawn in FIG. 85;

FIG. 86B is an axial cross section of a disc and the first step in the insertion of the intradiscal component drawn in FIG. 86A;

FIG. 86C is an axial cross section of a disc and the embodiment of the invention drawn in FIG. 86B;

FIG. 86D is an axial cross section of a disc and the embodiment of the invention drawn in FIG. 86C;

FIG. 87A is an oblique view of a novel intradiscal device;

FIG. 87B is a sagittal cross section of a portion of the spine, the embodiments of the invention drawn in FIGS. 75C and 87A;

FIG. 88A is a lateral view of an alternative embodiment of the invention drawn in FIG. 87A;

FIG. 88B is a sagittal view of a portion of the spine and the embodiment of the invention drawn in FIG. 88A;

FIG. 89A is a view of the head of a novel interference screw;

FIG. 89B is a view of the head of the embodiment of the interference screw drawn in FIG. 89A;

FIG. 90A is a lateral view of a portion of the spine with a dotted line representing an alternative embodiment of the invention drawn in FIG. 21A; and

FIG. 90B is an exploded lateral view of the spine in the embodiment of the invention drawn in FIG. 90A.

### DETAILED DESCRIPTION OF THE INVENTION

The methods and apparatus taught herein prevent excessive pressure on the Annulus Fibrosis (AF). In the preferred embodiment, a nucleus replacement (NR) is inserted from a lateral aspect of the lumbar spine at L4/L5 or above. A transpsoas technique is used in the preferred lateral approach to the lumbar spine. Identification of nerves within the psoas muscle allows the nerves to be protected and retracted anterior and posterior as necessary. A nerve stimulator may be used to help identify the nerves. Alternatively, the psoas muscle could be split with an endoscope that delivers or records electrical impulses. The myotomes of the extremities could be monitored to detect the electrical impulses. The novel NR could also be used from an anterior approach to the cervical spine. The dam or barrier embodiments of the invention may be used in conjunction with natural as well as artificial discs.

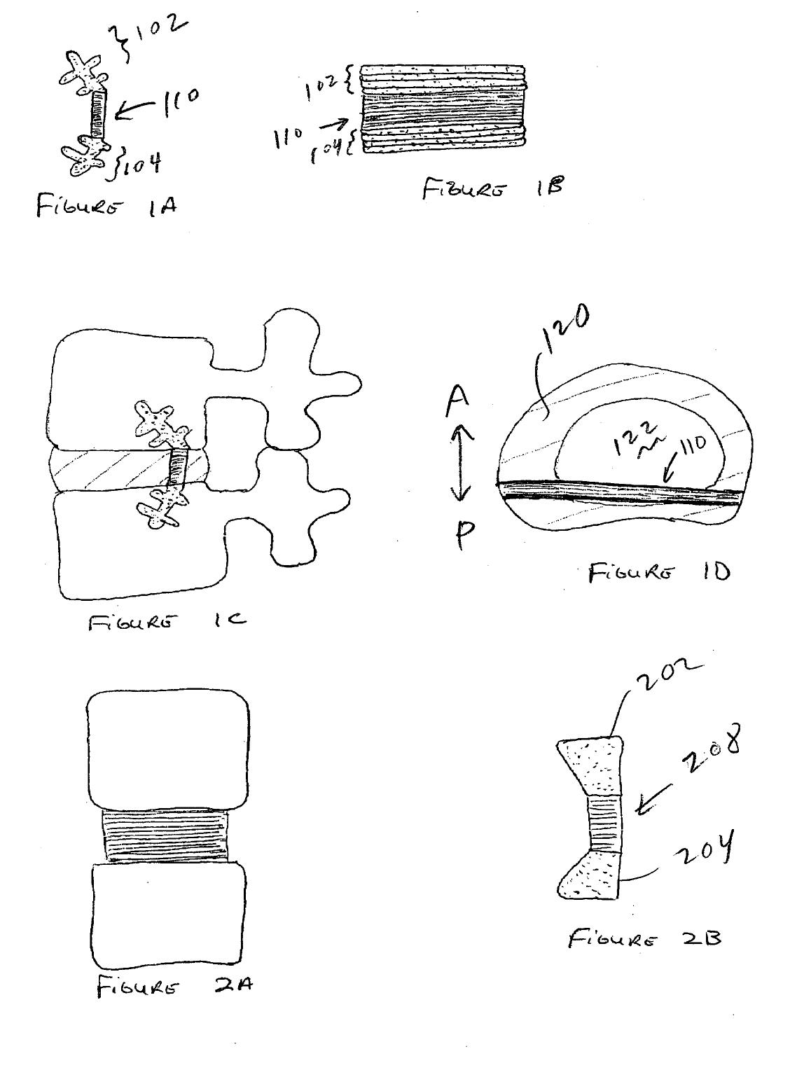

FIG. 1A is a lateral view of a “curtain” annulus augmentation device similar to the annulus augmentation devices taught in my U.S. Pat. No. 6,371,990, the entire content of which is incorporated herein by reference. Areas 102, 104 of the drawing represent spikes composed of titanium or other suitably rigid biocompatible material(s). The spikes slide into slots that are machined into the vertebrae. The section 110 of the device represents a dam, preferably constructed of a flexible, braided or mesh material such as nylon or Dacron. FIG. 1B is an anterior view of the embodiment of the invention drawn in FIG. 1A.

FIG. 1C is a lateral view of the spine and the embodiment of the invention drawn in FIG. 1A. The spikes fit into slots that were machined into the vertebrae. The flexible component of the device lies within the disc space. FIG. 1D is an axial cross section of the disc and device drawn in FIG. 1C. The AF is indicated at 120, and the Nucleus Pulposus (NP) or disc space is shown at 122. According to a preferred placement, the flexible component 110 of the device lies anterior to the posterior AF. The device prevents intradiscal devices, such as NRs, from applying pressure to the posterior portion of the AF.

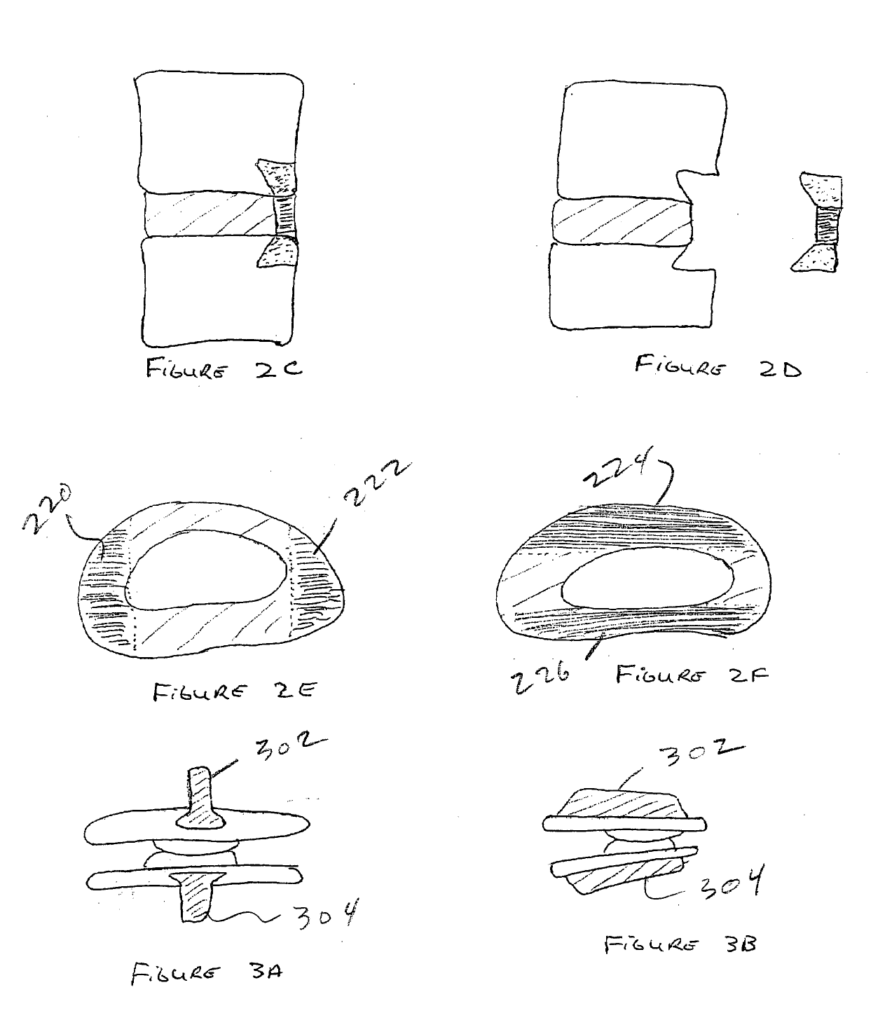

FIG. 2A is an anterior view of the spine and an alternative embodiment with the disc not included to better illustrate the device. FIG. 2B is a lateral view of the embodiment of the device drawn in FIG. 2A. The portions 202, 204 of the drawing represent portions of allograft vertebral bodies. The area 208 represents allograft AF. The allograft device is positioned in the spine as illustrated in FIGS. 1C and 1D. The partial dove-tail like allograft bones fit into slots machined into the vertebrae. Suitable materials other than allograft may alternatively be used.

FIG. 2C is an anterior view of a segment of a cadaver spine. The drawing illustrates one methods of harvesting the device drawn in FIG. 2A. The donor unit includes a portion of the vertebra above and below the disc and the AF between the vertebrae. FIG. 2D is an exploded anterior view of the cadaver spine drawn in FIG. 2C.

FIG. 2E is an axial cross section of a cadaver disc. The areas 220, 222, 224, 226 represent donor AF units. The drawing illustrates methods to harvest the left and right portions of the AF. FIG. 2F is an axial cross section of a cadaver disc. The drawing represents methods to harvest the anterior and posterior portions of the AF.

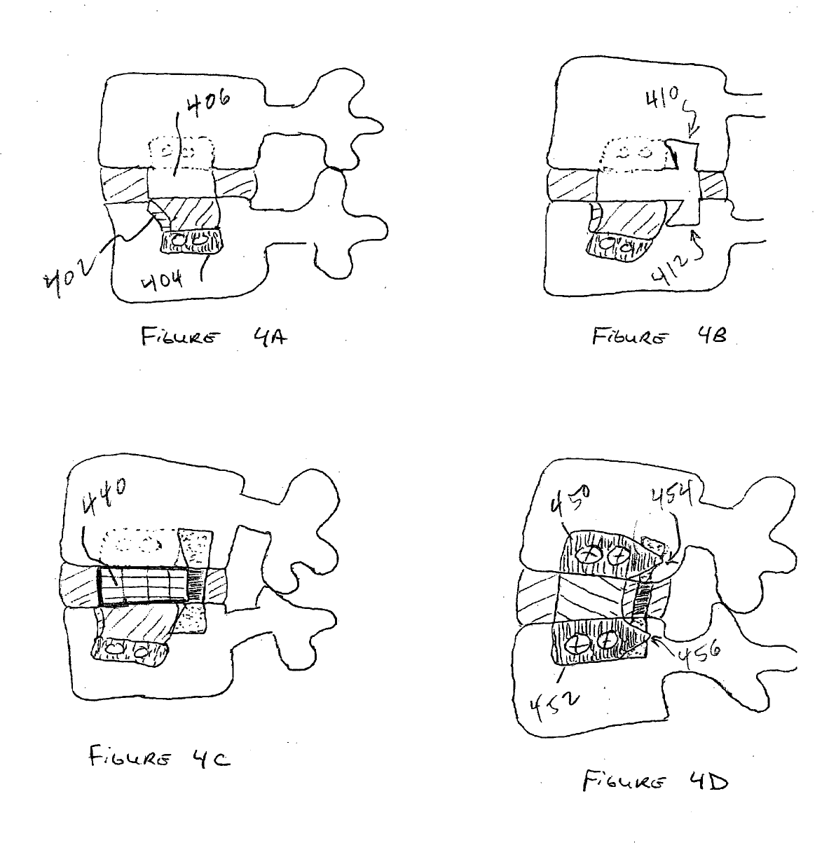

FIG. 3A is an anterior view of a novel ADR that may be placed anterior to the device drawn in FIG. 1C. Note that the keels 302, 304 may be made of allograft bone, and may be held in the ADR with screws or other fasteners (not shown). Bone keels facilitate the ingrowth of bone from the vertebrae. ADRs with bone keels would be easier to revise than ADRs with metal keels. FIG. 3B is a lateral view of the ADR drawn in FIG. 3A. Bone keels are applicable to ADRs and implants other than the one depicted in FIGS. 3A and 3B.

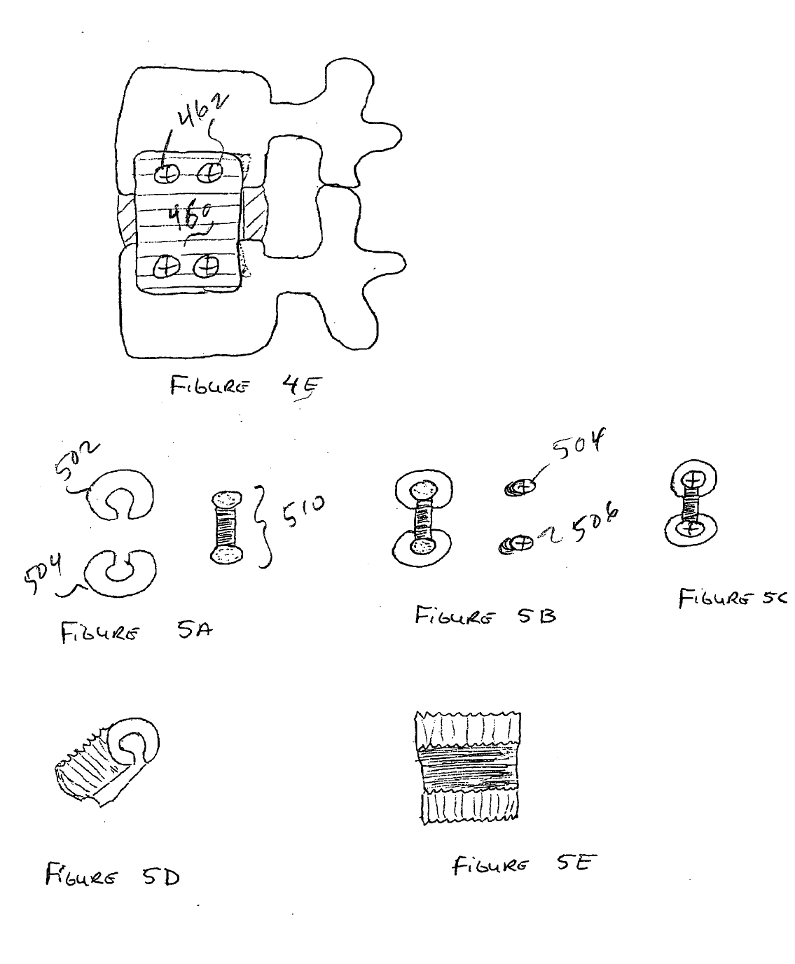

FIG. 4A is a lateral view of the spine used to illustrate methods to insert intradiscal devices as taught in my co-pending U.S. patent application Ser. No. 10/421,434, the entire content of which is incorporated herein by reference. This co-pending application teaches osteotomy of a vertebra to aid intradiscal device insertion. FIG. 4A shows an AF flap 402 and osteotomized bone 404 pulled inferiorly to create a window 406 into the disc space. FIG. 4B is a lateral view of the spine drawn in FIG. 4A. The vertebrae have been machined at 410, 412 to accept the embodiment of the invention drawn in FIG. 2B.

FIG. 4C is a lateral view of the spine, the embodiment of the device drawn in FIG. 2B, and a NR 440. The NR lies anterior to the embodiment of the invention drawn in FIG. 2B. FIG. 4D is a lateral view of the spine. Plates 450, 452 such as those taught in my co-pending application 10/421,434 are used to hold the osteotomized bone fragment in position. Extensions 454, 456 from the posterior portions of the plate also help hold the annulus augmentation device in place.

FIG. 4E is a lateral view of the spine an alternative embodiment of a plate 460 that holds the vertebral bone fragment in position. A resorbable plate is preferably used in this embodiment of the invention. A resorbable plate also temporarily immobilizes the spine while the bone grows into the device drawn in FIG. 2B. Resorbable screws 462 may be used with a resorbable plate. Alternatively, the spine could be temporarily immobilized with pedicle screws.

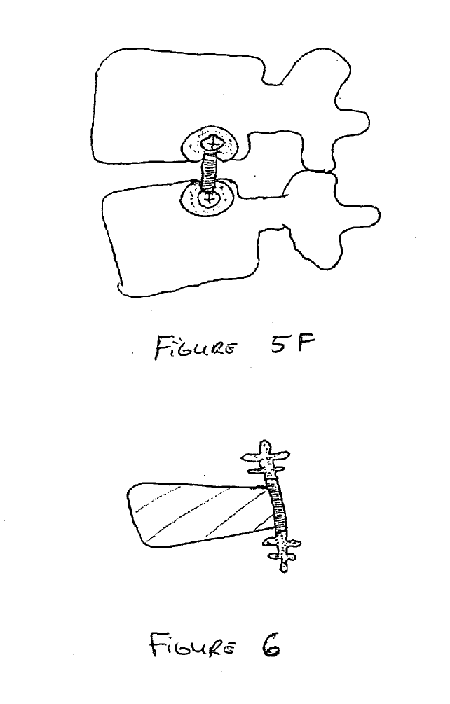

FIG. 5A is an exploded lateral view of an alternative embodiment of a flexible component 510 having ends shaped to fit into slots in screws 502, 504 placed transversely across a disc space. FIG. 5B is an exploded lateral view of the embodiment of the device drawn in FIG. 5A. Set screws 504, 506 may be used to hold the flexible component in the screws. FIG. 5C is a lateral view of the assembled device drawn in FIG. 5B. FIG. 5D is an oblique view of the screw (502, 504) drawn in FIG. 5A. FIG. 5E is an anterior view of the assembled device drawn in FIG. 5C. FIG. 5F is a lateral view of the spine and the embodiment of the device drawn in FIG. 5E. FIG. 6 is lateral view of the device drawn in FIG. 1A incorporated into the posterior aspect of the NR.

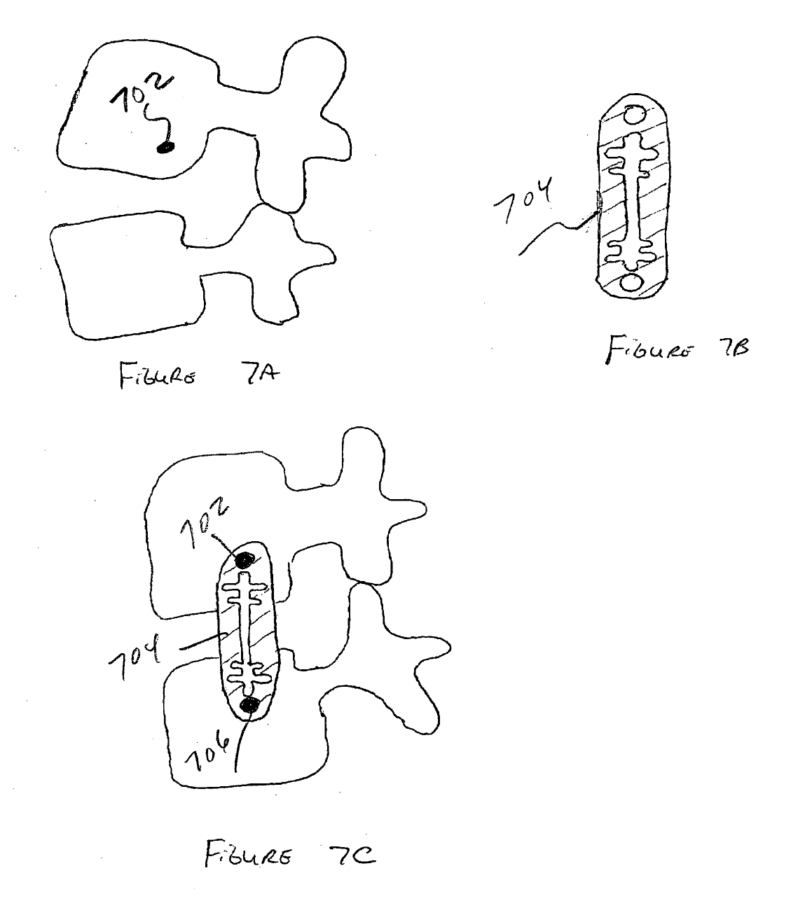

FIG. 7A is a lateral view of the spine. The drawing illustrates placement of a guide wire 702 into the vertebra. Fluoroscopy, CT scan, or other navigational devices may be used to facilitate placement of the guide wire. FIG. 7B is a lateral view of a guide 704 that fits over the guide wire drawn in FIG. 7A. The guide is used to as template to machine slots in the vertebrae. Drills or saws may be used to create the slots. FIG. 7C is a lateral view of the spine and the guide drawn in FIG. 7B. The drawing illustrates the use of two guide wires 702, 706 to hold the template on the spine.

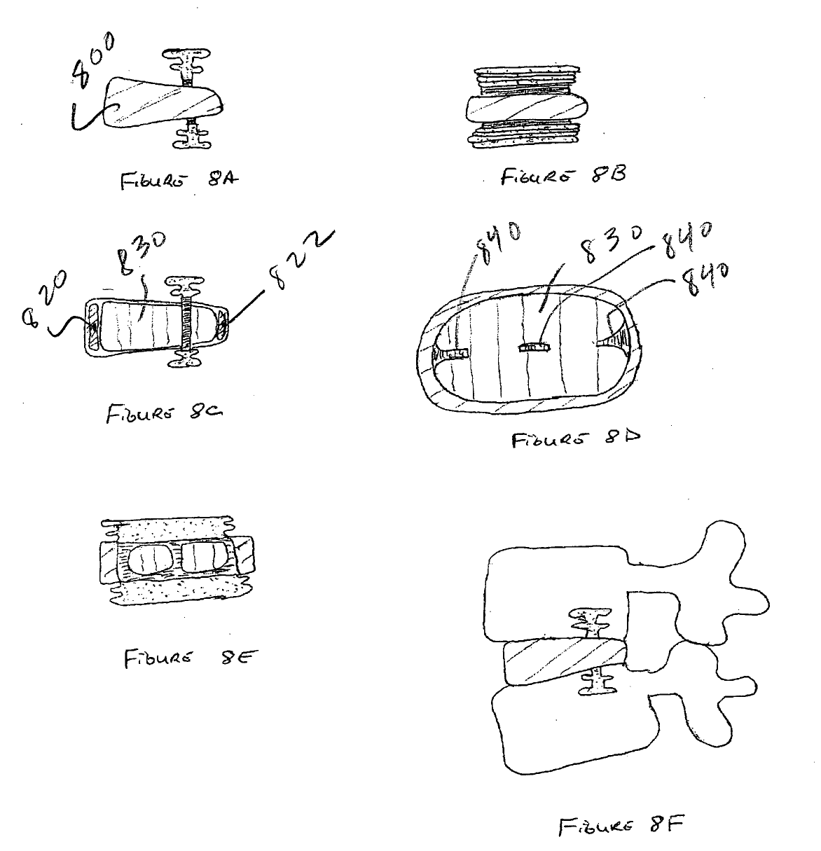

FIG. 8A is a lateral view of an alternative embodiment of the NR drawn in FIG. 6, wherein the embodiment of the invention drawn in FIG. 1A is incorporated into the body of a NR 800. FIG. 8B is an anterior view of the embodiment of the NR drawn in FIG. 8A. FIG. 8C is sagittal cross section of the embodiment of the NR drawn in FIG. 8A. The flexible retention component between the spikes lies within the NR. The NR incorporates bands as taught in my U.S. Pat. No. 6,419,704, the entire content of which is incorporated herein by reference. The flexible retention component lies within the bands or belts 820, 822. The area of the drawing 830 represents a cushion material such as hydrogel, polyurethane, or other suitable compressible or resilient substance.

FIG. 8D is an axial cross section of the embodiment of the NR drawn in FIG. 8A. The flexible retention member 840 is fenestrated to allow the cushion material to pass on either side of the retention member. Alternatively, the flexible member could separate the cushion area of the device into separate compartments. FIG. 8E is a coronal cross section of the embodiment of the NR drawn in FIG. 8A. FIG. 8F is a lateral view of the spine and the embodiment of the NR drawn in FIG. 8A.

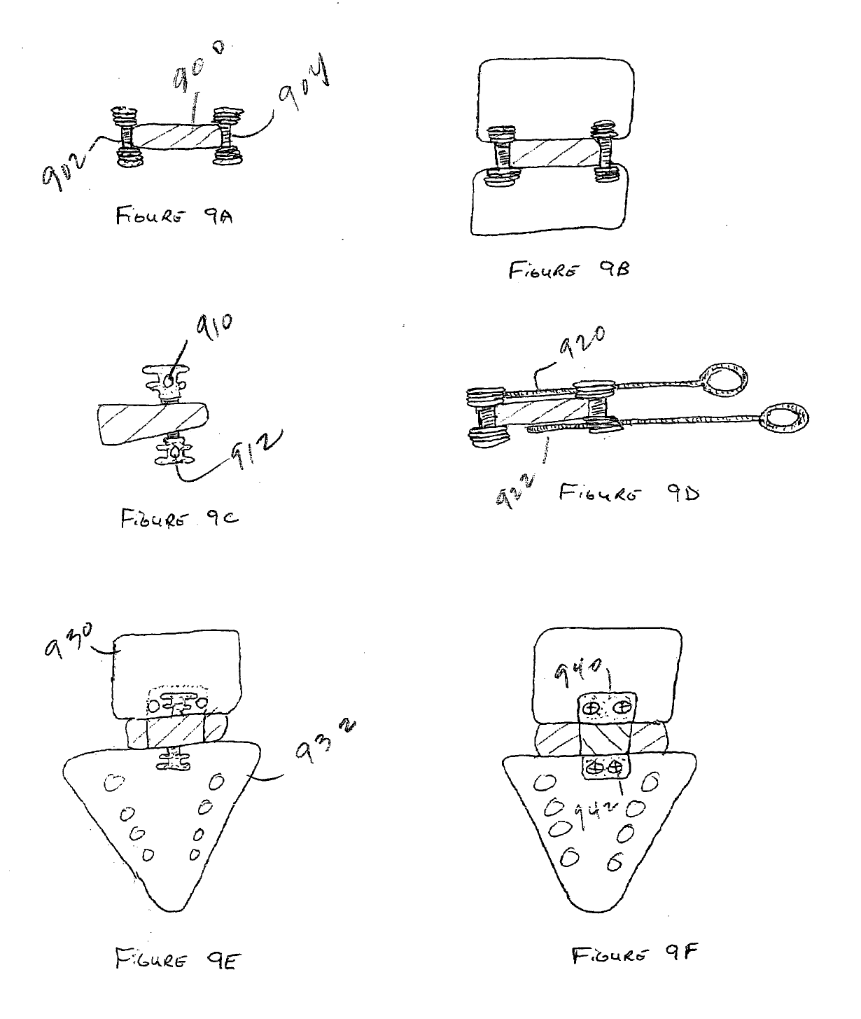

FIG. 9A is an anterior view of an alternative embodiment wherein flexible retention members 902, 904 are incorporated into the outside of an NR 900. For example, the retention members may lie on the left and right sides of the NR or anterior and posterior. FIG. 9B is an anterior view of the spine and the embodiment of the NR drawn in FIG. 9A. The NR was inserted from a lateral approach to the spine. The ends of the retention components fit into slots machined from the lateral side of the spine.

FIG. 9C is a lateral view of an embodiment similar to that drawn in FIG. 9A, wherein the retention members have holes 910, 912 to accept tools to insert the NR. FIG. 9D is an anterior view of the embodiment of the NR drawn in FIG. 9A. The drawing illustrates the use of wires 920, 922 to insert the NR. The wires pass through the holes in the near retention members to the retention members on the far side of the NR. Wires are used to push the retention members into the machined slots in the vertebrae FIG. 9E is an anterior view of the L5 vertebra 930, the sacrum 932, and an alternative embodiment of an NR with a retention member (within the NR) that courses from anterior to posterior. The NR has been inserted through an osteotomized flap on the anterior side of the spine. The dotted line represents the edges of the vertebral fragment. FIG. 9F is an anterior view of the spine drawn in FIG. 9E. Plates 940, 942 are shown holding the NR and the vertebral fragment on the spine.

FIG. 9G is an axial cross section of the embodiment of the NR drawn in FIG. 9E. The flexible retention member separates left and right cushion compartments 940, 942 of the NR. FIG. 9H is a lateral view of the L5 vertebra 950, the sacrum 952, and the embodiment of the NR drawn in FIG. 9E. Although in this and other embodiments the retention member is shown coursing centrally through the disc area, other placements are possible depending upon spinal level, weaknesses in the AF, or other factors.

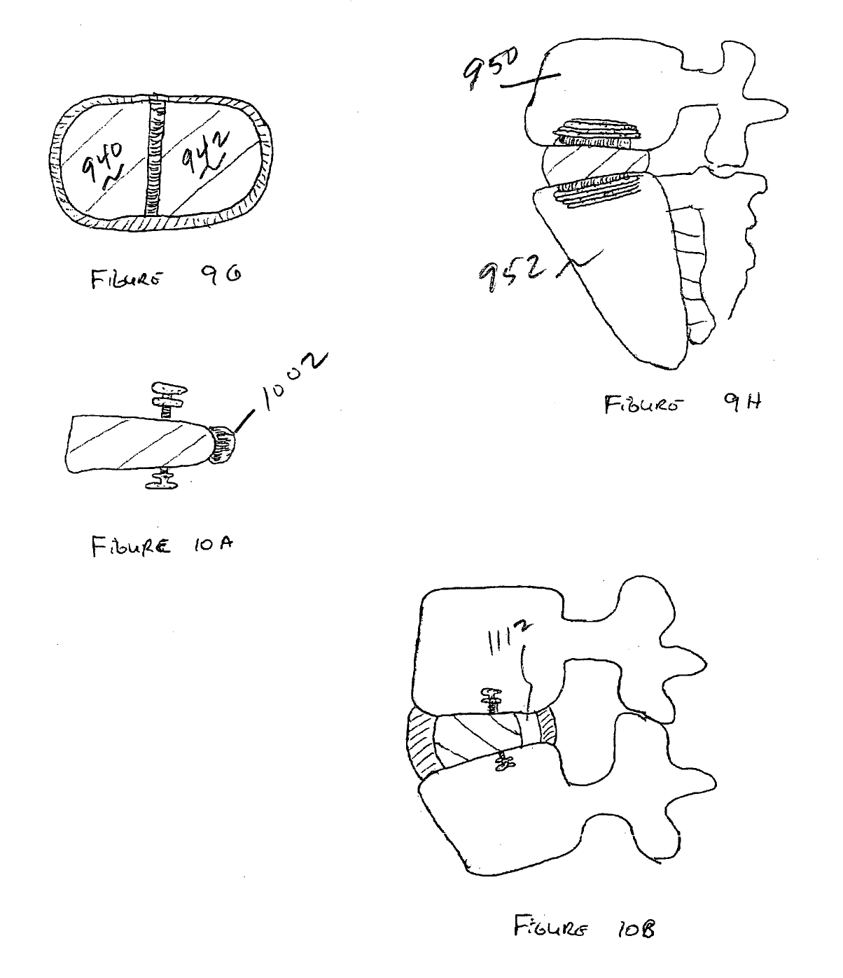

FIG. 10A is a lateral view of a version of the NR drawn in FIG. 8A that incorporates a balloon 1002 on the posterior aspect of the device. The balloon may be temporarily inflated to place the NR anterior to the posterior portion of the AF. The balloon may be deflated after placing the NR in the disc space. Deflating the balloon creates a space between the NR and the posterior portion of the AF. Alternatively, removable instruments may be placed between the NR and the posterior portion of the AF.

FIG. 10B is a lateral view of the spine and the embodiment of the NR drawn in FIG. 8A. A space 1112 can be seen between the NR and the posterior portion of the AF. The properly positioned NR is unlikely to apply pressure to the posterior portion of the AF.

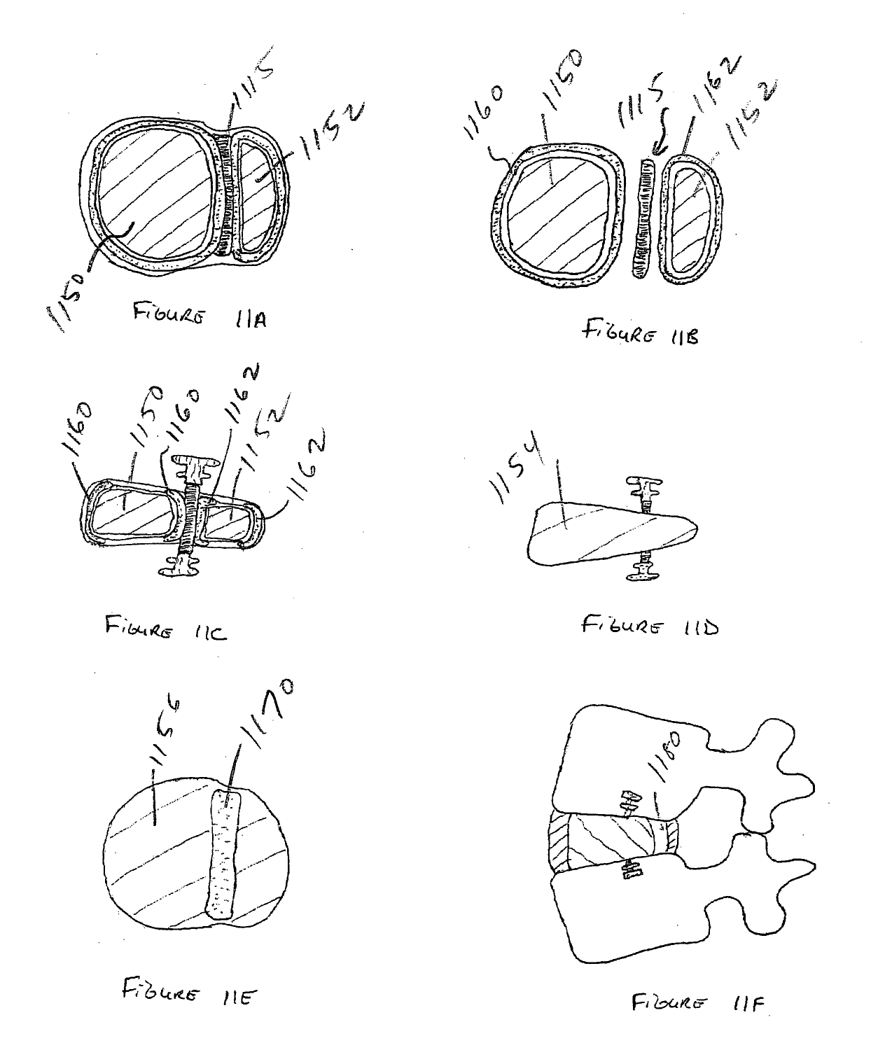

FIG. 11A is an axial cross section an alternative embodiment of the invention wherein a flexible retention component 1115 lies between separate anterior and posterior cushion components. The anterior cushion component is preferably larger than the posterior component in the preferred embodiment of the device, and both cushion components are preferably surrounded by the bands or belts taught in my '704 patent. The flexible bands may be made of a relatively inelastic material such as Dacron or nylon. The band or belts surround cushion material such as hydrogel or polyurethane. Other suitable materials may alternatively be used and this invention is not limited in this regard.

The anterior, posterior, and retention components may be dip-molded together. An elastic material, such as that used in cardiac balloon catheters, is used in the dip mold of the preferred embodiment of the device. The cushion material may be fully cured before insertion of the NR into the disc space. The disc space could be mechanically distracted prior to inserting the NR. Alternatively, the cushion material could cure in-situ. The radial belts around the anterior and posterior components cause the device to expand in a superior to inferior direction more than they expand in a radial direction. The NR could expand by injection of in-situ curing material or by the absorption of fluids. The dotted area of the drawing represents the relatively inelastic belts. The areas 1150, 1152, 1154, 1156 represent cushion material.

FIG. 11B is an exploded axial cross section of the embodiment of the invention drawn in FIG. 11A prior to surrounding the components with an elastic material. FIG. 11C is a sagittal cross section of the embodiment of the NR drawn in FIG. 11A. The radial belts are depicted at 1160, 1162.

FIG. 11D is a lateral view of the embodiment of the NR drawn in FIG. 11A. FIG. 11E is a view of the top of the embodiment of the NR drawn in FIG. 11A. The anterior cushion component is larger than the posterior cushion component in the preferred embodiment. Area 1170 represents the portion of the retention component that fits into the slots machined into the vertebrae. FIG. 11F is a lateral view of the spine and the embodiment of the NR drawn in FIG. 11A. The drawing illustrates a space 1180 between the NR and the posterior portion of the AF.

FIG. 12A is an axial cross section of an alternative embodiment of the invention wherein the retention member separates cushion components 1202, 1204. Both cushion components are surrounded by relatively inelastic bands or belts 1206, 1208. The components are held together by an elastic material 1210. The NR could be used to replace the cervical NP or the L5/S1 NP. FIG. 12B is a lateral view of the embodiment of the NR drawn in FIG. 12A. The retention member courses from the anterior to posterior portion of the NR. FIG. 12C is a view of the top of the embodiment of the device drawn in FIG. 12B. FIG. 12D is a lateral view of the L5 vertebra 1220, the sacrum 1222, and the embodiment of the NR 1224 drawn in FIG. 12A.

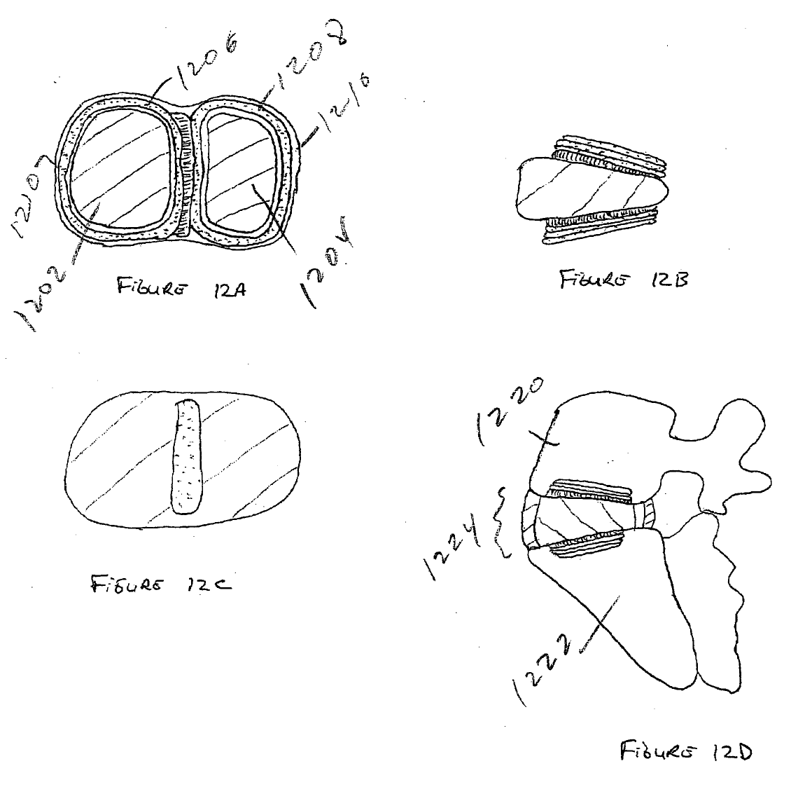

FIG. 13A is an oblique view of an alternative embodiment of the invention including a machined graft that is preferably harvested from a donor spine. The device includes a piece of donor Annulus Fibrosus (AF) 1302 with cylindrical pieces of donor vertebra 1304, 1306 attached to either side of the AF. The bone pieces are preferably 2 to 16 mm in diameter and 5 to 35 mm in length. The AF piece is preferably 2 to 40 mm wide and 5 to 20 mm tall. The allograft AF could be cylindrical shaped. The most preferred graft is 7-8 mm in diameter with bone components that are 10-15 mm long and an AF component that is 8-16 mm long.

FIG. 13B is an oblique view of an alternative embodiment of the invention drawn in FIG. 13A wherein the bone ends have at least one flat surface along their sides. FIG. 13C is an oblique view of an alternative embodiment of the invention drawn in FIG. 13B wherein the bone ends have at least one partially concave groove along their sides. FIG. 13D is an oblique view of an alternative embodiment of the invention wherein more than one side of the bone ends is flat.

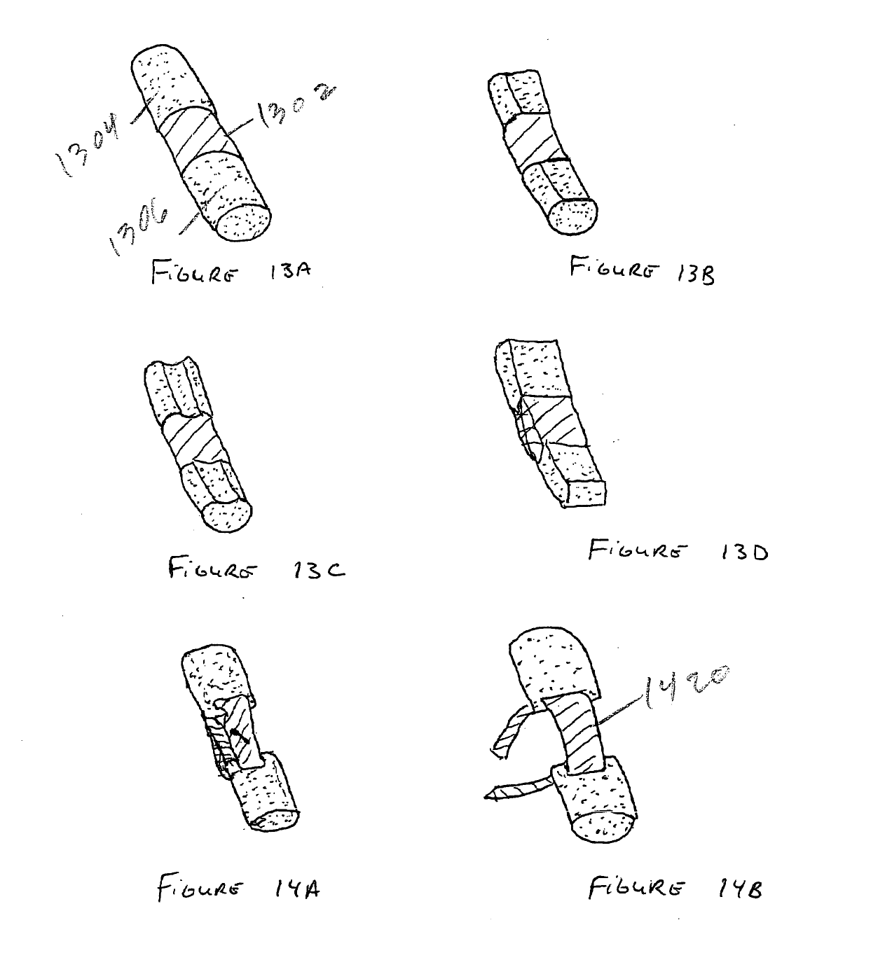

FIG. 14A is an oblique view of an alternative embodiment of the invention drawn in FIG. 13A. As taught in FIG. 5G of my U.S. Pat. No. 6,878,167, the allograft device could be constructed of tissues that are not natural to the spine. For example, tendon, ligament, fascia, or other soft tissue could be looped through holes in the ends of two pieces of bone. The allograft bones could be machined from long bones such as the humerus, radius, ulna, femur, tibia, fibula, the bones of the hands or feet including the metacarpals, metatarsals, or phalanges, or bones from the spine, pelvis or other location.

FIG. 14B is an oblique view of the embodiment of the invention drawn in FIG. 14A. The allograft soft tissue 1420 has been passed through holes in the allograft bone. The ends of the allograft soft tissue may be overlapped and fastened together. For example, the ends of the allograft soft tissue could be sutured together. Autograft tissue could be used in alternative embodiments of the invention. Bone pieces with natural soft tissue between the bone components could be harvested from areas of the body other than the spine. For example, small joints such as the joints in the hand, wrist, elbow, feet, ankle, knee, shoulder, hip, or other joint may be harvested with attached ligaments may be used in other embodiments of the invention.

FIG. 15 is an oblique view of an alternative embodiment of the invention drawn in FIG. 13A. The allograft device has been machined from a piece of bone 1502 with its tendon 1504 attached to the bone. For example, the bone could be harvested from the calcaneus with a portion of the Achilles tendon. Alternative tissues could be harvested such as a portion of the patella with attached quadriceps tendon or patellar tendon.

FIG. 16 is an alternative embodiment of the invention drawn in FIG. 15. The device is machined from allograft soft tissue, such as tendon or ligament. FIG. 17 is an alternative embodiment of the invention drawn in FIG. 16. The device is cut from a sheet of allograft soft tissue, such as fascia. FIG. 18 is an alternative embodiment of the invention drawn in FIG. 13A. One bone component 1802 has been threaded.

FIG. 19 is an alternative embodiment of the invention drawn in FIG. 19. One bone component 1902 has been threaded. Teeth have been machined on the other piece of bone 1904. The slopes of the sides of the teeth may differ to facilitate insertion but to resist extrusion from a direction opposite to the direction of insertion.

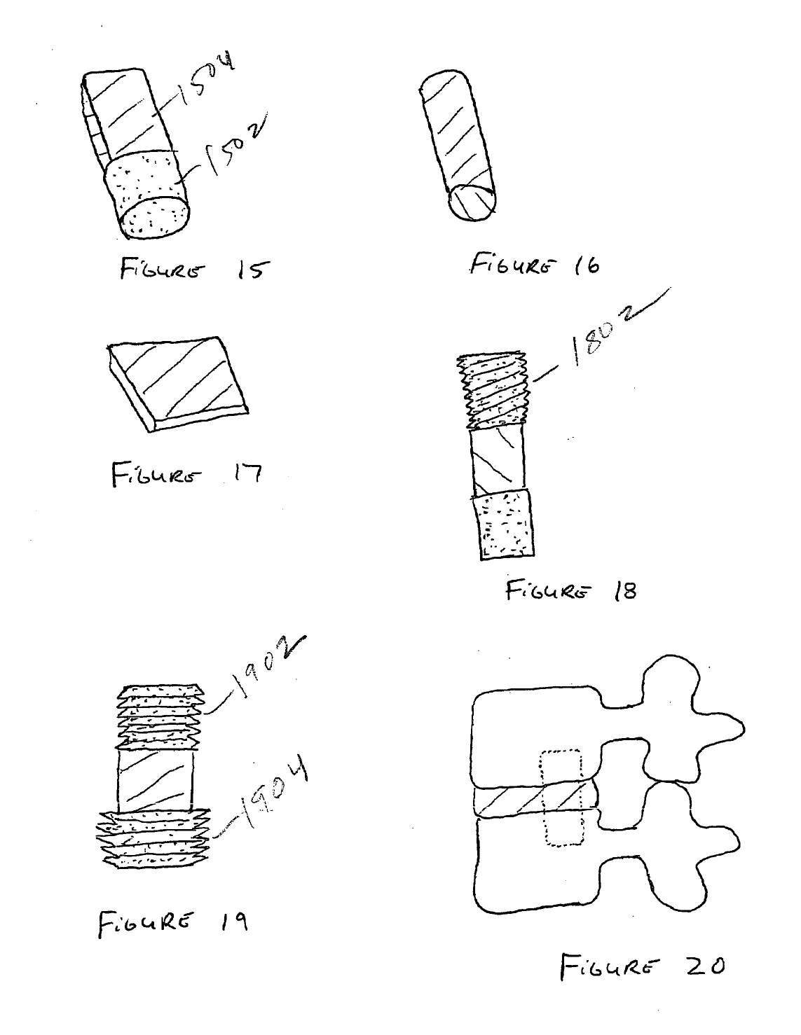

FIG. 20 is a lateral view of the spine. The dotted line represents a preferred allograft harvest site. The spinal allograft devices are preferably harvested from the anterior or lateral portion of the spine. The anterior and lateral portions of the AF are generally thicker and less degenerative than the posterior portion of the AF.

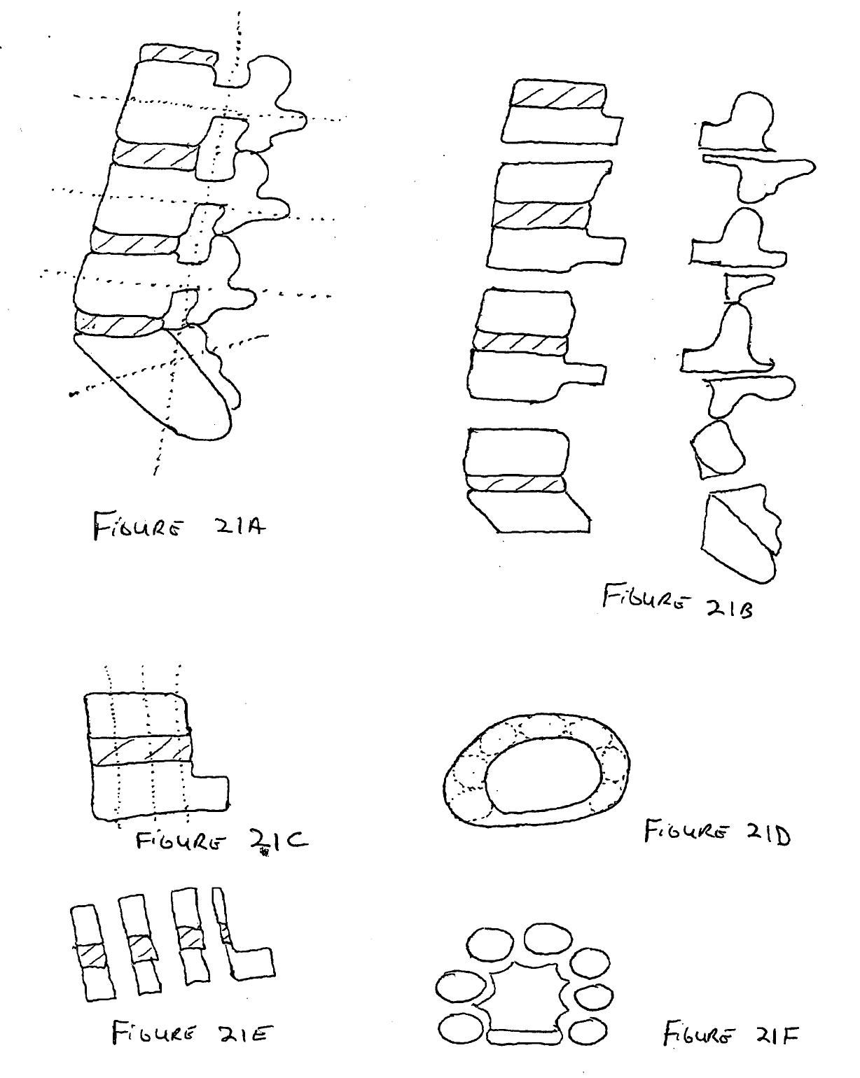

FIG. 21A is a lateral view of a portion of the lumbar spine. The dotted lines represent the preferred locations to cut a donor spine. FIG. 21B is an exploded lateral view of the spine drawn in FIG. 21A. The posterior elements have been separated from the vertebral bodies and discs. The vertebral bodies have been bisected in the axial plane. Each disc includes half of the vertebral body above the disc and half of the vertebral body below the disc.

FIG. 21C is a lateral view of one disc unit drawn in FIG. 21B. The dotted lines represent the preferred series of cuts to obtain multiple machined devices per disc unit. FIG. 21D is an axial cross section through the disc drawn in FIG. 21C. The dotted lines represent the preferred outline of the machined allograft devices. The drawing illustrates harvest of seven grafts per disc unit. Alternative number of grafts could be harvested per disc unit. The grafts may be harvested by a “hole saw” attachment to a drill. The grafts are preferably cut from the disc unit using CNC machines. FIG. 21E is an exploded lateral view of the embodiment of the invention drawn in FIG. 21C. FIG. 21F is an exploded axial view of the embodiment of the invention drawn in FIG. 21D.

FIG. 22A is an axial cross section of a disc donor unit (disc with portions of the vertebra above and below the disc). The dotted lines represent alternative sites to cut the disc unit. FIG. 22B is an exploded axial cross section of the donor disc unit drawn in FIG. 22A. Twelve allograft implants were harvested from this donor disc unit. The grafts are rectangular in shape. The AF was harvested in two layers to increase the yield of implants per donor disc unit.

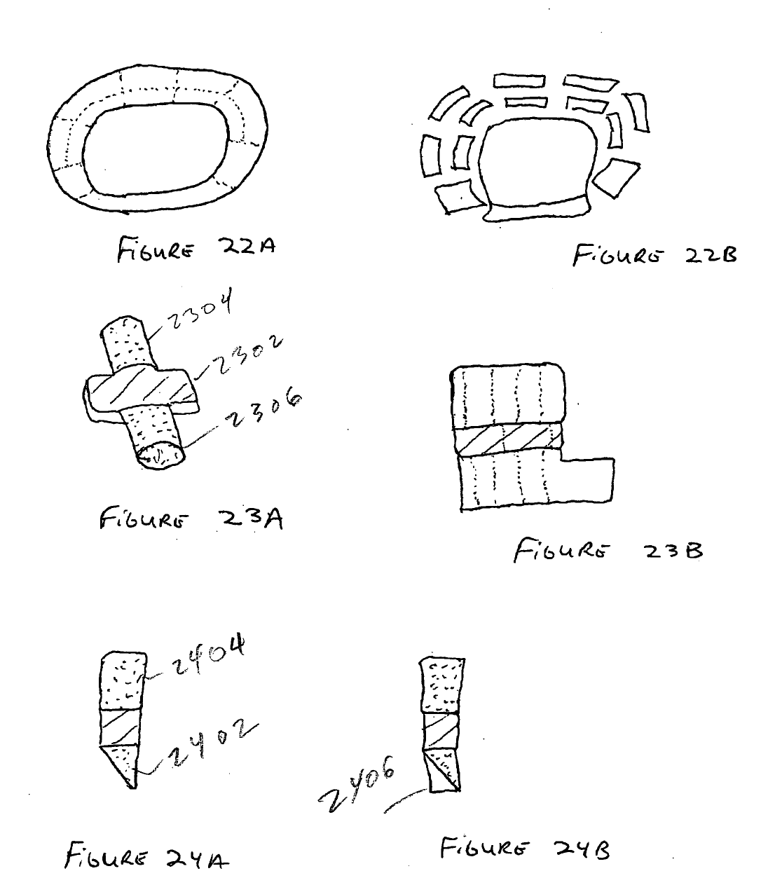

FIG. 23A is an oblique view of an alternative embodiment of the invention wherein the AF 2302 is wider than the bone components 2304, 2306. FIG. 23B is a lateral view of a donor disc unit. The dotted lines represent the preferred places to cut the disc unit to create the donor shape drawn in FIG. 23A.

FIG. 24A is a lateral view of the embodiment of the invention drawn in FIG. 13A. The bone component 2402 on the bottom of the graft is smaller than the bone component 2404 on the top of the graft. FIG. 24B is a lateral view of the embodiment of the invention drawn in FIG. 24A. An additional piece of bone 2406 has been pinned to the smaller bone component. Pins made of bone may be used to fasten the pieces of bone.

FIG. 25 is a partial sagittal cross section of a portion of the lumbar spine and an embodiment of the invention taught in FIG. 5G of my U.S. Pat. No. 6,878,167. The donor graft has been placed into holes drilled into the vertebra above and below the disc space. The holes have been drilled into the posterior aspect of the vertebral bodies. The graft is held in place with interference screws 2502, 2504.

My U.S. Pat. No. 6,245,107 teaches attachment of devices to prevent recurrent HNP, and to correct defects created in the vertebra above and below the disc space (FIG. 7B). My U.S. Pat. No. 6,371,990 (FIGS. 3-4) teaches augmentation of the AF by attaching a device with a flexible central component to the vertebra above and below the disc. The device is anchored to holes in the vertebrae above and below the disc. The invention may be used to reconstruct the disc following Microlumbar Discectomy (MLD), reconstruct the disc following insertion of an intradiscal device (such as a Nucleus Replacement, Total Disc Replacement, fusion cage, or other natural or synthetic device), or to treat tears of the AF. The invention prevents the Nucleus Pulposus (NP) or the intradiscal device from extruding through defects in the AF. The invention enables the retention of contained NP during MLD. The invention may be used on the anterior, lateral, or posterior portions of the cervical, thoracic, or lumbar spine.

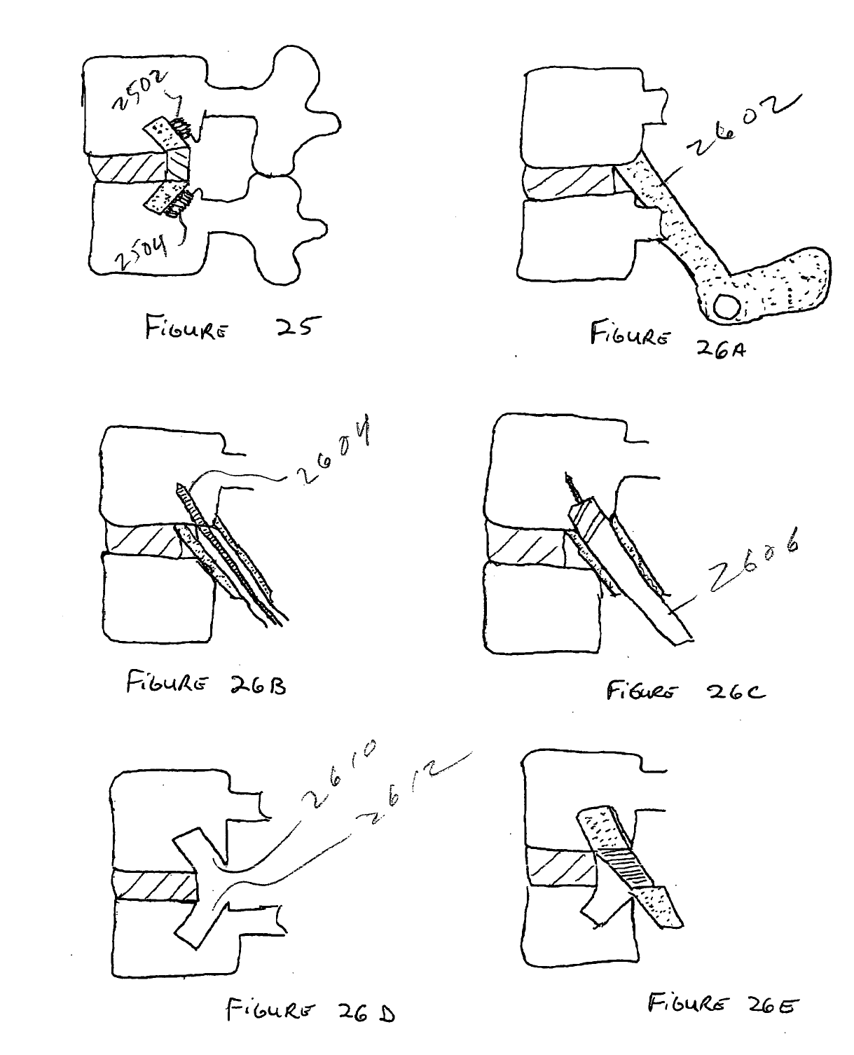

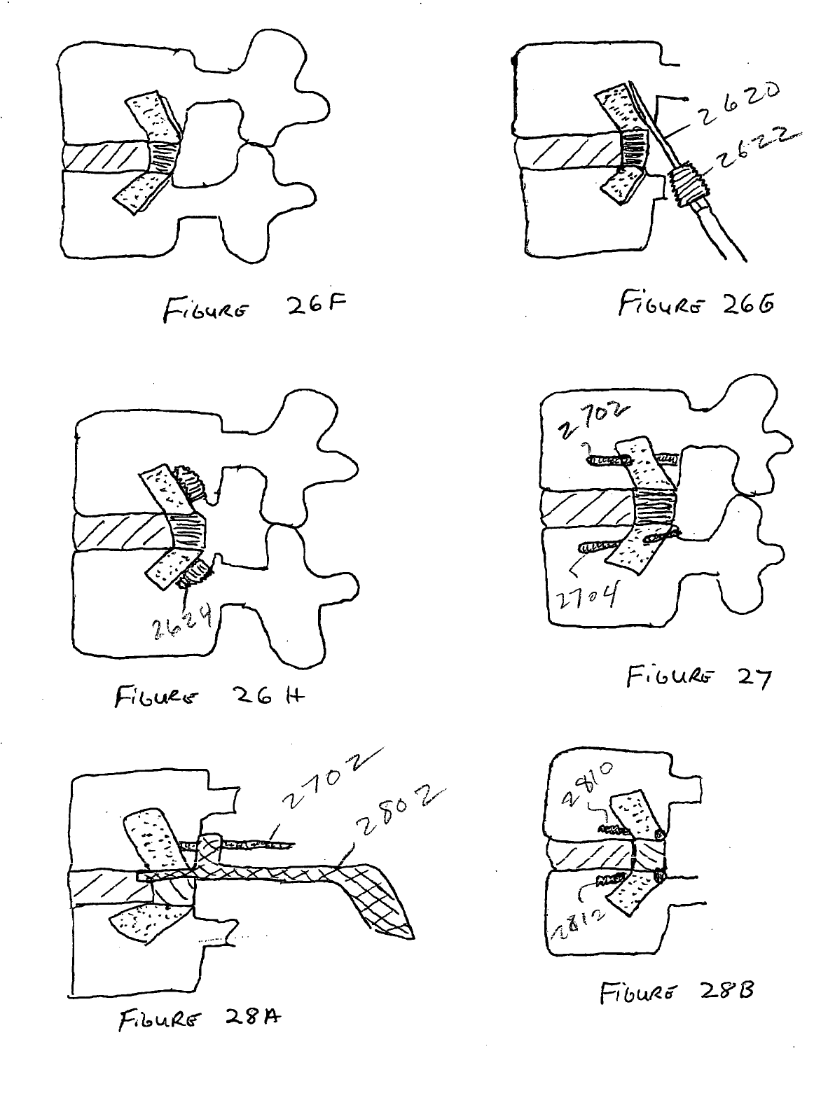

FIG. 26A is a lateral view of the spine and the first step of a method of inserting the device drawn in FIG. 13A. A drill sleeve 2602 is used to protect the nerves and to direct a drill or K-wire into the vertebra above the disc. FIG. 26B is a sagittal cross section of the spine and the embodiment of the invention drawn in FIG. 26A. A K-wire 2604 has been drilled into the vertebra above the disc. The position of the K-wire may be placed with the aid of Fluoroscopy, CT, and/or computer aided navigational technology or robotic technology.

FIG. 26C is a sagittal cross section of the spine and the embodiment of the invention drawn in FIG. 26B. A cannulated drill 2606 or reamer has been passed over the K-wire. FIG. 26D is a sagittal cross section of a portion of the spine. Holes 2610, 2612 have been drilled into the vertebra above and below the disc using the technique taught in FIG. 26C.

FIG. 26E is a sagittal cross section of a portion of the spine and the embodiment of the invention drawn in FIG. 13A. The first end of the device has been inserted into hole 2610 in the vertebra above the disc. FIG. 26F is sagittal cross section of a portion of the spine and the embodiment of the invention drawn in FIG. 13A. The ends of the device have been placed into the holes 2610, 2612 in the vertebra above and below the disc.

FIG. 26G is a sagittal cross section of the spine and the embodiment of the invention drawn in FIG. 26F. A K-wire 2620 has been placed into the hole in the vertebra above the disc. The K-wire is adjacent to the first bone end of the graft. A cannulated interference screw 2622 has been placed over the K-wire. The K-wire guides the cannulated interference screw along the side of the bone end of the device. FIG. 26H is a lateral view of a portion of the spine and the embodiment of the invention drawn in FIG. 26G. A second interference screw 2624 has been inserted to fasten the second end of the device.

A torque measuring screwdriver could be used to insert the interference screws. The torque measuring screwdriver could assure the interference screw fits tightly into the vertebra. The interference screw could be made of metal such as titanium or allograft bone, or a bio-resorbable material. Suitable bio-resorbable materials associated with this and alternative embodiments of the invention include polylactic acid (PLA), polyglycolic acid (PGA), poly (ortho esters), poly(glycolide-co-trimethylene carbonate), poly-L-lactide-co-6-caprolactone, polyanhydrides, poly-n-dioxanone, and poly(PHB-hydroxyvaleric acid). The interference screw may have anti-back out features, including those drawn in FIGS. 89A & 89B. The interference screws are preferably 2-9 mm in diameter and 3 to 15 mm in length. For example, the screws may be 5 mm in diameter and 10 mm long. Interference screws may be obtained from DePuy Inc. (Warsaw, Ind.).

FIG. 27 is a partial sagittal cross section of a portion of the spine and an alternative fixation method. Pins 2702, 2704 are placed through portions of the vertebrae and the bone ends of the device taught in FIG. 13A. Alternative fixation or anchor technology well know to Orthopaedic Surgeons who practice Sports Medicine may be used. For example the fixation technology used in ACL reconstruction, shoulder reconstruction, or ankle reconstruction may used to secure the spinal implant.

FIG. 28A is a partial sagittal cross section of a portion of the spine and the embodiment of the invention drawn in FIG. 27. A guide 2802 is used to direct the pin 2702 through the end of the spinal implant. The first end of the guide cooperates with features in the spinal implant to align the hole in the guide with the hole in the implant. For example, the end of the guide may straddle the spinal implant at the junction of the donor AF and donor bone. Alternatively, a navigational guidance system may be used to direct the cross pins. Navigational systems may be used to direct cross pins in other embodiments of this technology. For example, navigational technology may be used in cross pinning ACL grafts. The cross pins may be made of metal, bone, or bio-resorbable materials. Materials that remodel into bone are used in preferred embodiments of the device.

FIG. 28B is a partial sagittal cross section of a portion of the spine and an alternative embodiment of the invention wherein the holes that accept the cross pins 2810, 2812 or screws may be seen at the junction of the bones and the AF. The invention eliminates the need for guides or navigational systems.

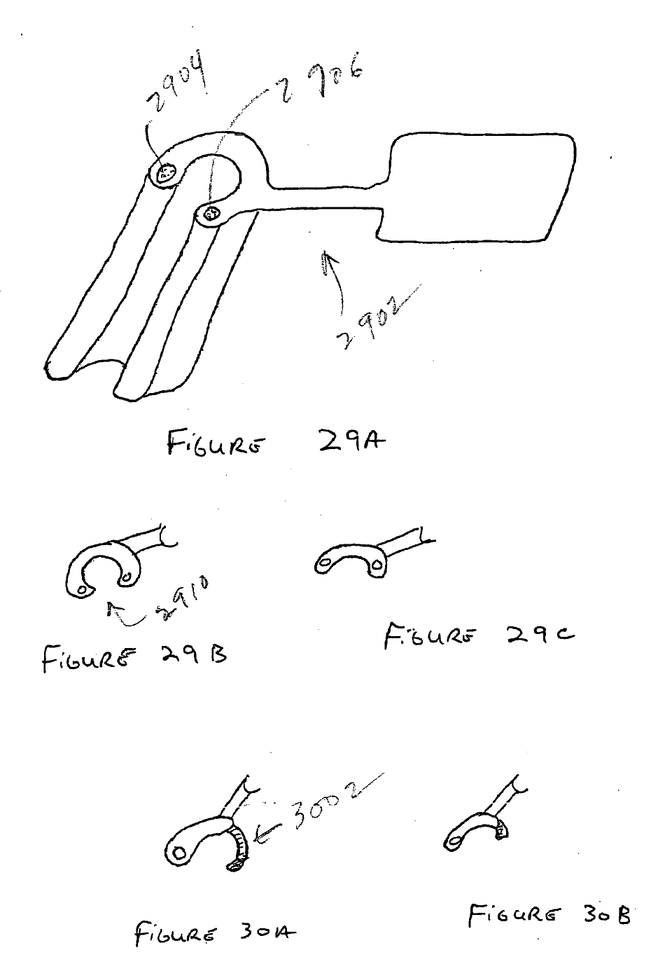

FIG. 29A is an oblique view of a novel guide according to the invention. The device may be used to protect the nerves, guide instruments used to create the holes in the vertebrae, and to guide insertion of the implant drawn in FIG. 13A. The instrument 2902 has holes 2904, 2906 that pass through the guide. K-wires may be passed through the guide and into the vertebrae to stabilize the guide.

FIG. 29B is a view of the top of a portion of the embodiment of the invention drawn in FIG. 29A. The guide has an opening 2910 along the side of the device. The slot shaped opening allows visualization of the instruments as they are passed into and out of the guide. The guide may surround over 180 degrees of the instruments and graft. The “constrained” embodiment of the guide holds prevents the instruments or graft from falling out of the side of the guide.

FIG. 29C is a view of the top of a portion of an alternative embodiment of the invention wherein the guide surrounds less than 180 degrees of the instruments or grafts that are passed through the guide. The “unconstrained” embodiment of the guides facilitates removal of the guide once the graft has been placed into the spine.

FIG. 30A is a view of the top of a portion of an alternative guide that has a retractable component 3002. The retractable component may be extended to surround more than 180 degrees of the instruments. FIG. 30B is a view of the top of a portion of the embodiment of the invention drawn in FIG. 30A. The guide has been drawn in its “contracted” form. The retracted configuration of the guide facilitates removal of the guide after insertion of the implant into the spine.

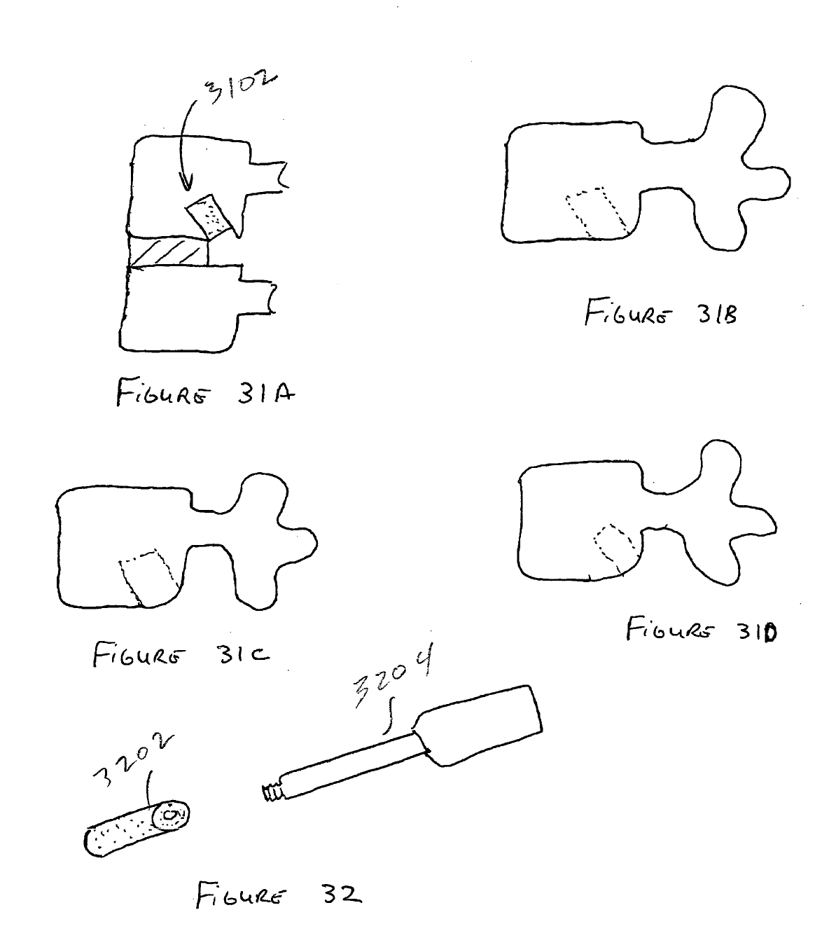

FIG. 31A is a partial sagittal cross section of a portion of the spine. The drawing illustrates one preferred location 3102 of the holes that may be drilled into the vertebrae. The holes may be drilled through the vertebral endplates (VEPs) just anterior to the posterior portion of the vertebral bodies. FIG. 31B is a lateral view of a vertebra. The dotted lines indicate a second preferred location of the holes that receive the device drawn in FIG. 13A. The holes may drilled through the VEPs further anterior to the junction of the VEPs and the posterior portion of the vertebral bodies.

FIG. 31C is a lateral view of a vertebra. The dotted lines indicate an alternative location of the holes that drilled into the vertebral bodies. The holes may extend into the VEPs and the posterior vertebral bodies. FIG. 31D is a lateral view of a vertebra. The dotted lines indicate an alternative location of the holes drilled into the vertebra. The holes may enter a larger portion of the posterior vertebral bodies than the VEPs.

FIG. 32 is an exploded oblique view of a plug 3202 and an inserter tool 3204 according to the invention. The plug may be threaded on to the inserter tool. Alternative mechanisms may be used to temporarily connect the plug and the insertion tool. The plug may be temporarily placed into the first hole drilled in the first vertebra. The plug prevents bleeding from the first hole while drilling of the second hole. The plug sits flush with the opening of the first hole so as avoid obstructing the instruments used to create the second hole. The plug is the length of the first hole. The plug may be made of material or coated with material that facilitates clotting of the blood. For example, the plug could be made of thrombin impregnated collagen.

FIG. 33 is a sagittal cross section of a portion of the spine and an alternative embodiment of the invention drawn in FIG. 26H. The first interference screw 3302 has been placed anterior to the graft. Anterior placement of the interference screw prevents the screw from backing out into the spinal canal.

FIG. 34 is an axial cross section of the disc following MID. A defect 3410 remains in the AF at the site of the herniated nucleus pulposus (HNP). Prior-art techniques include removal of generous amounts of the NP from the disc to help prevent extrusion of the NP from the defect in the AF. Removal of the NP accelerates disc degeneration.

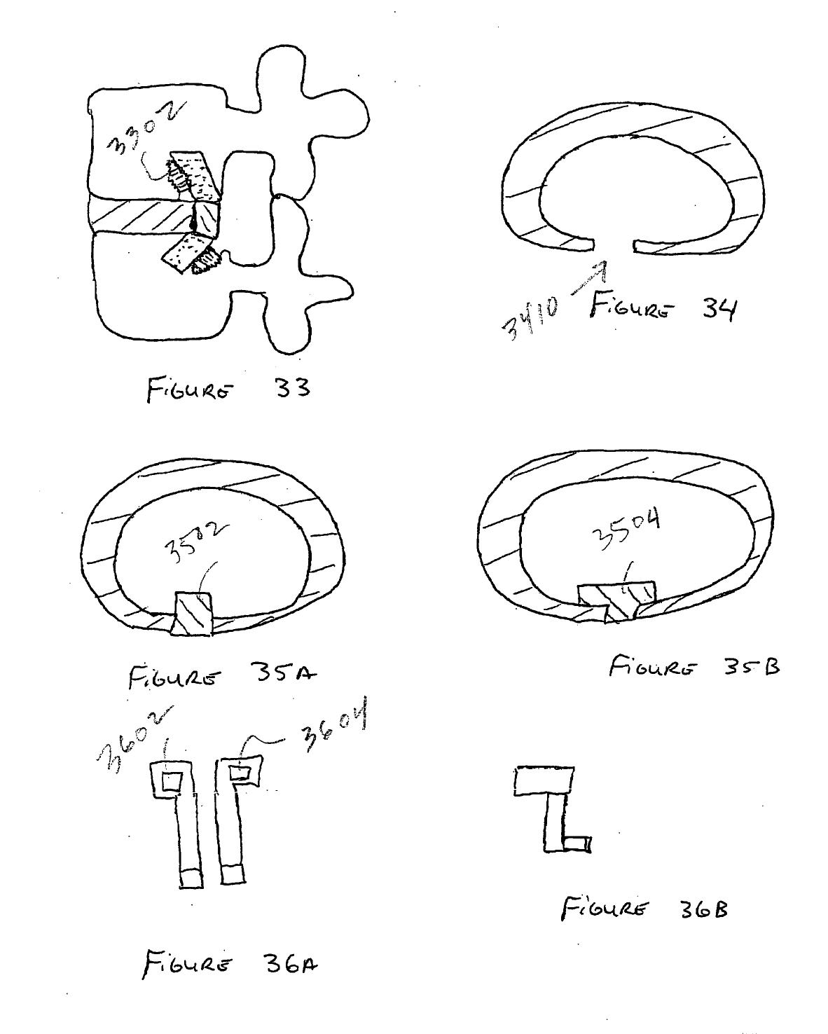

FIG. 35A is an axial cross section of a disc and the embodiment of the invention drawn in FIG. 33. The allograft AF 3502 fills the opening from the MLD. The allograft AF is generally thicker than the adjacent host AF. The allograft AF was harvested from the thicker anterior or lateral portions of the donor AF. The invention enables surgeons to preserve the contained NP during MLD. FIG. 35B is an axial cross section of a disc and an alternative embodiment of the invention wherein the donor AF 3504 has been shaped to partially overlap the host AF. The overlap helps prevent extrusion of NP between the donor and host AF.

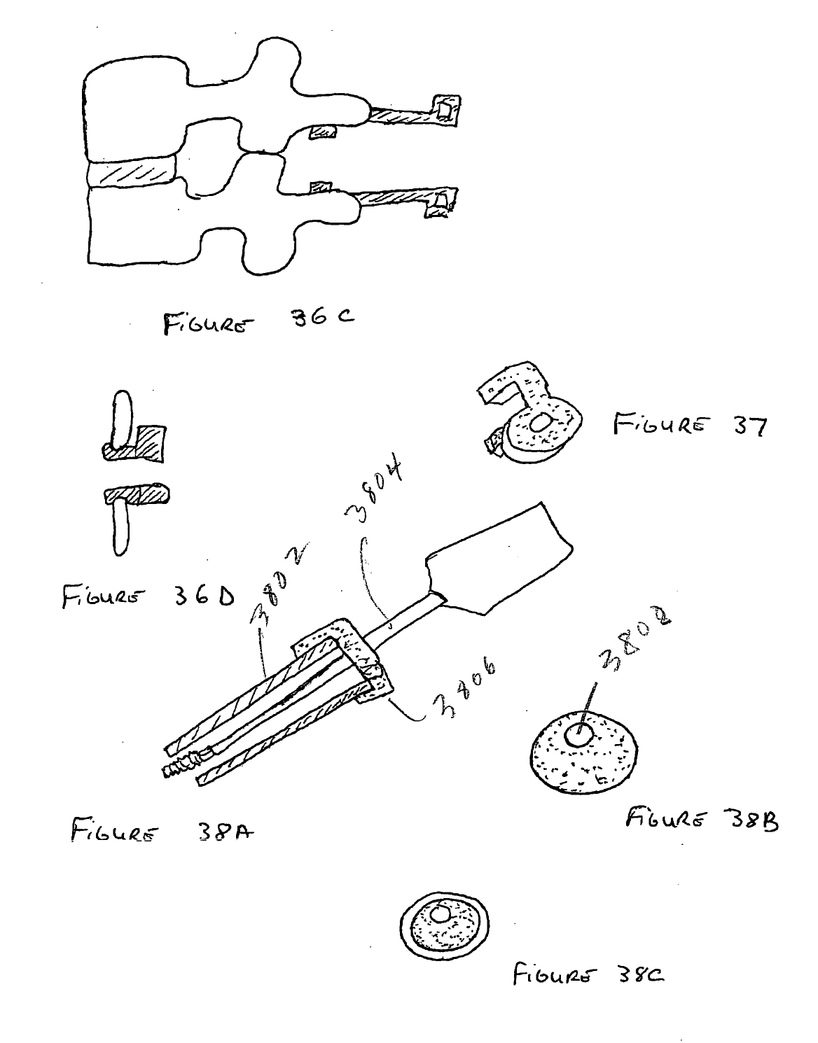

FIG. 36A is lateral view of novel distractor blades used to facilitate insertion of the graft drawn in FIG. 33. The squares 3602, 3604 at the tops of the blades represent square openings. The blades fit over arms of a distractor. For example, the blades may be placed over the arms of a McClough retractor (V. Mueller). The opposite ends of the blades fit between the spinous processes of the vertebrae.

FIG. 36B is a view of the cephalad side of the embodiment of the invention drawn in FIG. 36A. FIG. 36C is lateral view of a portion of the spine and the embodiment of the invention drawn in FIG. 36B. The tips of the blades are forced through the interspinous ligament. The distractor increases the size of the interlaminar window. The distractor also flexes the spine. The graft is generally fixed to a flexed spine. The graft may fail during spinal flexion if it is fixed to an extended spine. FIG. 36D is view of the dorsal aspect of adjacent spinuos processes and the tips of the distractor taught in FIG. 36C.

FIG. 37 is an oblique view of a novel instrument that may be used to help guide insertion of the interference screws. The instrument fits over the end of the sleeve drawn in FIG. 29A. Projections from the sides of the instrument align the instrument over the opening in the sleeve. The shaft of a screwdriver fits through the opening in the “cap” instrument.

FIG. 38A is a sagittal cross section of a sleeve 3802, a screwdriver 3804, and alternative embodiment of the cap instrument 3806 drawn in FIG. 37. FIG. 38B is a view of the top of the cap instrument drawn in FIG. 38A. The eccentric hole 3808, the sleeve, and the cap cooperate to direct interference screws parallel to and against the bone ends of the embodiment of the invention drawn in FIG. 33. FIG. 38C is a view of the bottom of the embodiment of the invention drawn in FIG. 38B.

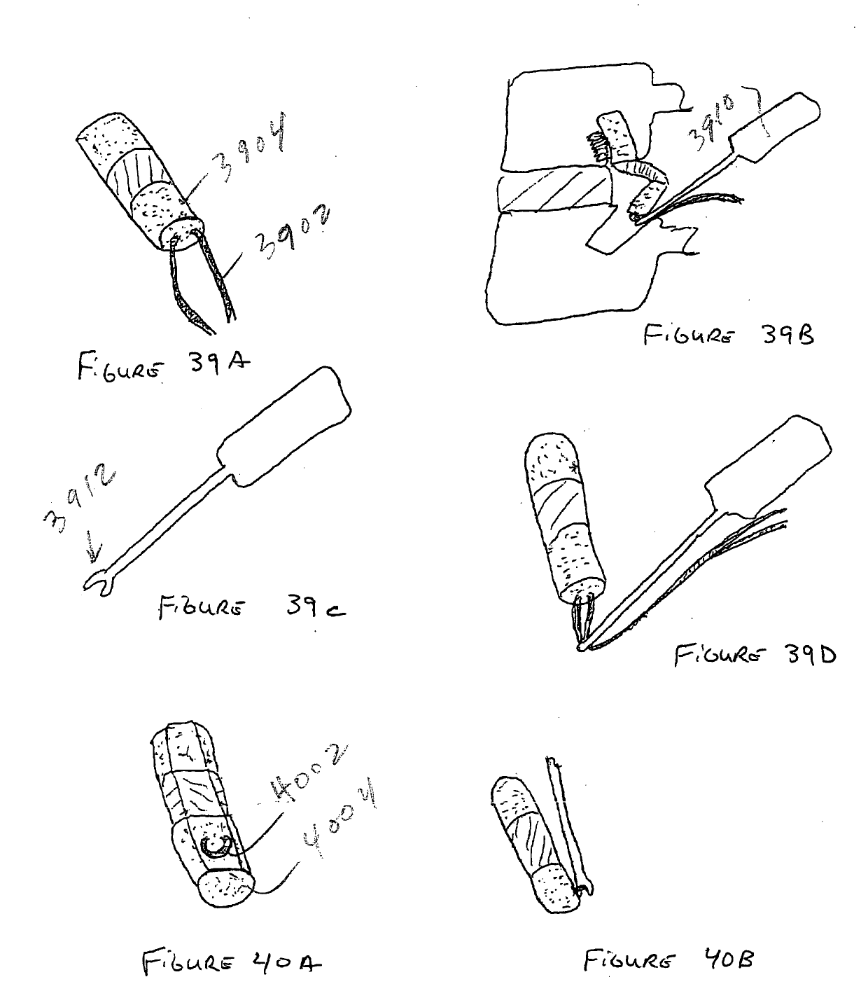

FIG. 39A is an oblique view of an alternative embodiment of the invention wherein a suture 3902 has been passed through a hole in the end of one of the bone components 3904. FIG. 39B is a sagittal cross section of a portion of the spine and the embodiment of the invention drawn in FIG. 39A. A tool 3910 may cooperate with the suture to direct the graft into the hole in the vertebra. FIG. 39C is a lateral view of the embodiment of the tool drawn in FIG. 39B. The tip 3912 of the tool is forked to fit over the sutures. FIG. 39D is an oblique view of the embodiments of the invention drawn in FIGS. 39A and 39C. The graft may be manipulated by applying tension to the sutures. The tool helps “pull” the graft into the hole drilled into the vertebra.

FIG. 40A is an oblique view of an alternative embodiment of the invention wherein a loop of suture 4002 has been passed through a hole in one of the bones 4004 of the graft. The suture may be made of resorbable or non-resorbable suture. FIG. 40B is a lateral view of the embodiments of the invention drawn in FIGS. 39C and 40A. The tip of the instrument fits into the loop in the graft. The instrument may be used to direct the graft into the hole in the vertebra.

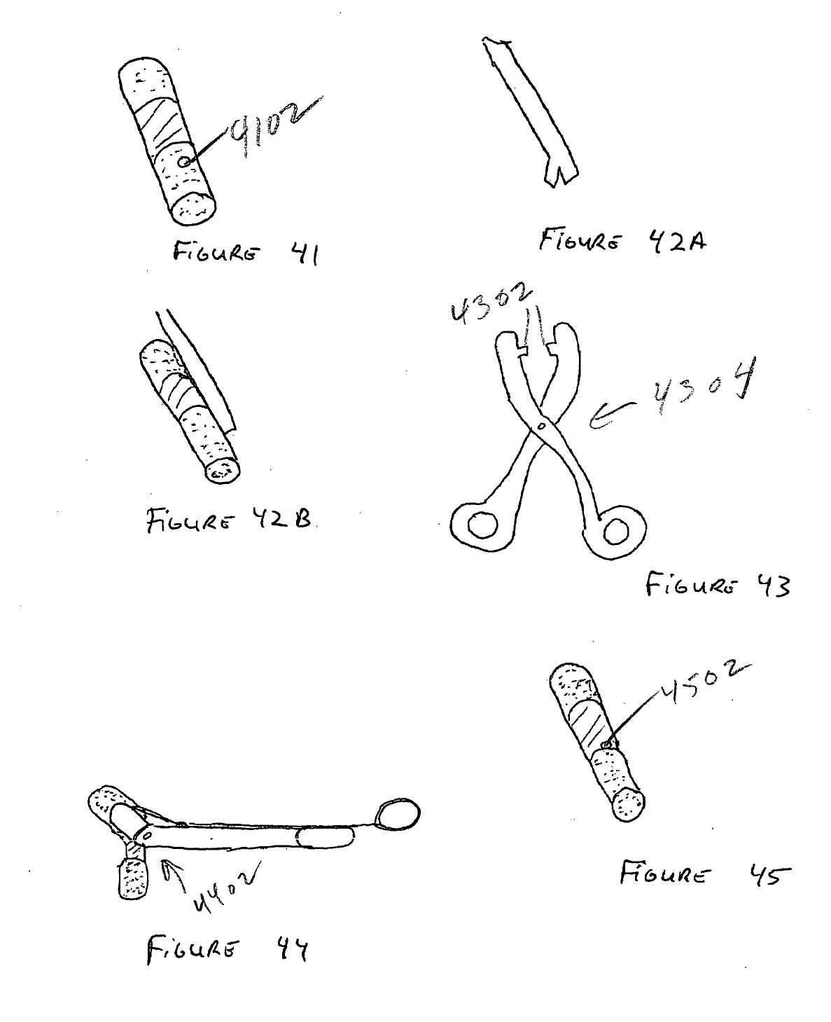

FIG. 41 is an oblique view of an alternative embodiment of the invention wherein a hole 4102 has been drilled into one or both pieces of bone.

FIG. 42A is a view of a portion of an alternative embodiment of the invention drawn in FIG. 39C. A projection from the tip of the instrument fits into the hole or holes in the graft. FIG. 42B is a lateral view of the embodiments of the graft drawn in FIGS. 41 and 42A. The instrument may be used to manipulate the graft.

FIG. 43 is a lateral view of an alternative embodiment of the invention drawn in FIG. 42A. Projections 4302 from the sides of the tips of the pliers-like instrument 4304 may fit into holes in the sides of the graft. The instrument may be used to grasp and manipulate the graft.

FIG. 44 is a lateral view of an alternative embodiment of the invention and a graft similar to that drawn in FIG. 13A. A hinge joint 4402 connects the tips of the instrument to the shafts of the instrument. A wire may be used to change the angle of the tips of the instrument relative to the shafts of the instrument. Alternatively, the instrument may have gearing that permits changing the angle formed between the tips of the instrument and the shafts of the instrument. The invention facilitates insertion of the bone ends of the graft into the holes drilled into the vertebrae.

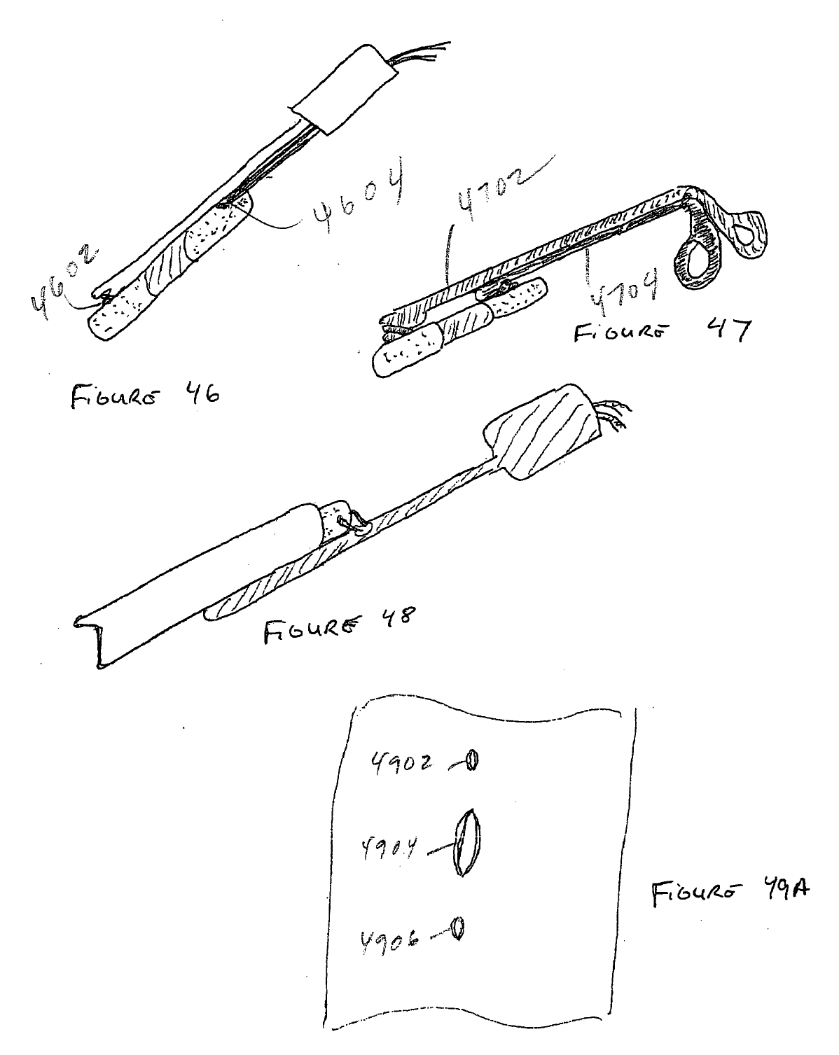

FIG. 45 is an oblique view of an alternative embodiment of the invention drawn in FIG. 41. A hole 4502 has been placed into the AF at the junction of the AF and the bone.

FIG. 46 is a lateral view of alternative embodiments of the inventions drawn in FIG. 39C and 40A. Suture loops 4602, 4604 have been placed into both pieces of bone. Tension is applied to the graft by pulling the sutures in opposite directions. The tip of the tool fits into the first suture loop. The tips of the second suture loop pass through the handle of the instrument.

FIG. 47 is a lateral view of an alternative embodiment of the invention drawn in FIG. 46. The novel instrument pulls the loops in opposite directions. The first arm 4702 of the instrument applies tension on the first suture loop. The second arm 4704 of the instrument applies tension on the second suture loop. FIG. 48 is a lateral view of embodiments of the invention drawn in FIG. 46 and 29A. The suture loops pass through the slot in the retractor sleeve as the graft is passed through the sleeve.

FIG. 49A is a view of a portion of the back of a patient. The three elliptical openings 4902, 4904, 4906 represent incisions. The larger central incision 4904 represents a MLD (Microscopic Lumbar Discectomy) incision. Instruments are passed through the smaller incisions above and below the MLD incision.

FIG. 49B is a partial sagittal cross section of the back, a muscle retractor 4902, the retractor sleeve 2902 drawn in FIG. 29A, and a drill bit 4904. The retractor blade is positioned within the MLD incision. The sleeve and drill bit pass from the incision below the MLD incision and into the MLD wound. The technique enables surgeons to place the bone ends of the graft more parallel to the long axis of the spine.

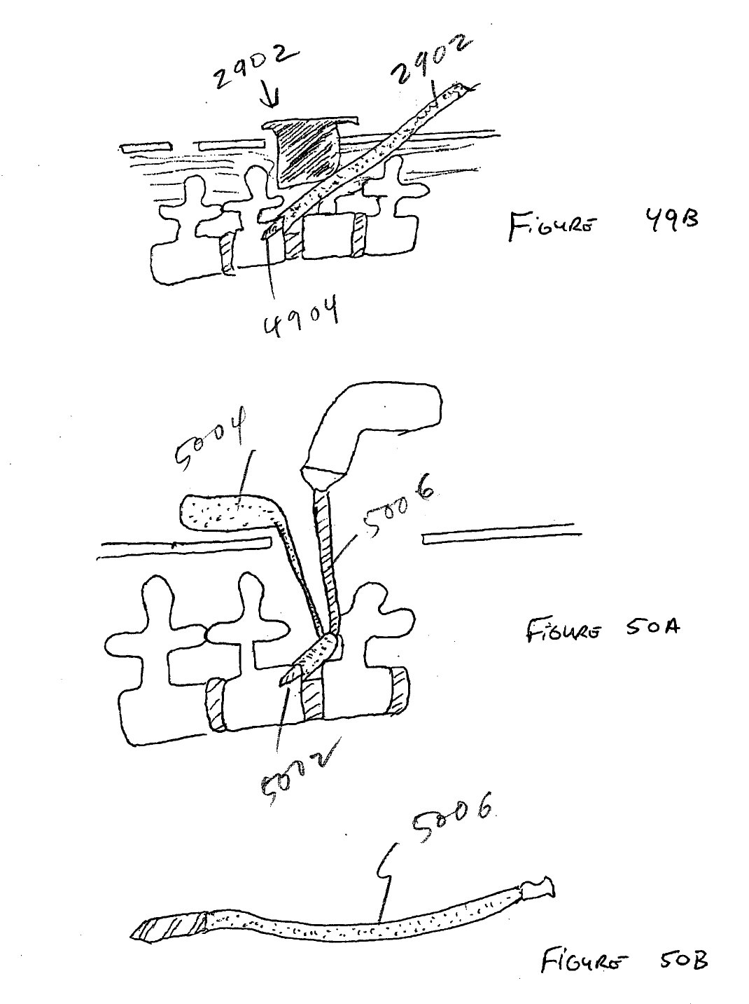

FIG. 50A is a partial sagittal cross section of the back and an alternative embodiment of the invention wherein a flexible drill bit 5002 is passed through a novel drill guide 5004. The invention enables surgeons to place the bone ends of the graft as described in the text of FIG. 49B through a single incision. The drill guide directs the drill bit and protects the nerves. FIG. 50B is a lateral view of the flexible drill bit drawn in FIG. 50A. The shaft 5006 of the drill bit is flexible.

FIG. 51 is a sagittal cross section of an alternative embodiment of the invention drawn in FIG. 33, wherein AF portion of the graft is longer than the patient's AF. The AF portion of the graft extends into the holes drilled into the vertebrae. FIG. 52 is a sagittal cross section of an alternative embodiment of the invention drawn in FIG. 51, wherein the AF portion of the graft is shorter than the patient's AF. The bone ends of the graft extend beyond the entrance to the holes drilled into the vertebrae.

FIG. 53 is an axial cross section of a disc and an alternative embodiment of the invention drawn in FIG. 35A wherein the graft 5302 is placed anterior to the opening in the patient's AF. The AF portion of the graft is wider than the opening in the patient's AF. The donor AF may be fixed to the patient's AF. For example, the AF components may be sutured, stapled, laser welded, or glued together. A biologic adhesive such as fibrin glue or other biocompatible adhesive such as supplied by BioDisc or BioGlue by Cyro-Life or NuCore by Spine Wave may be used.

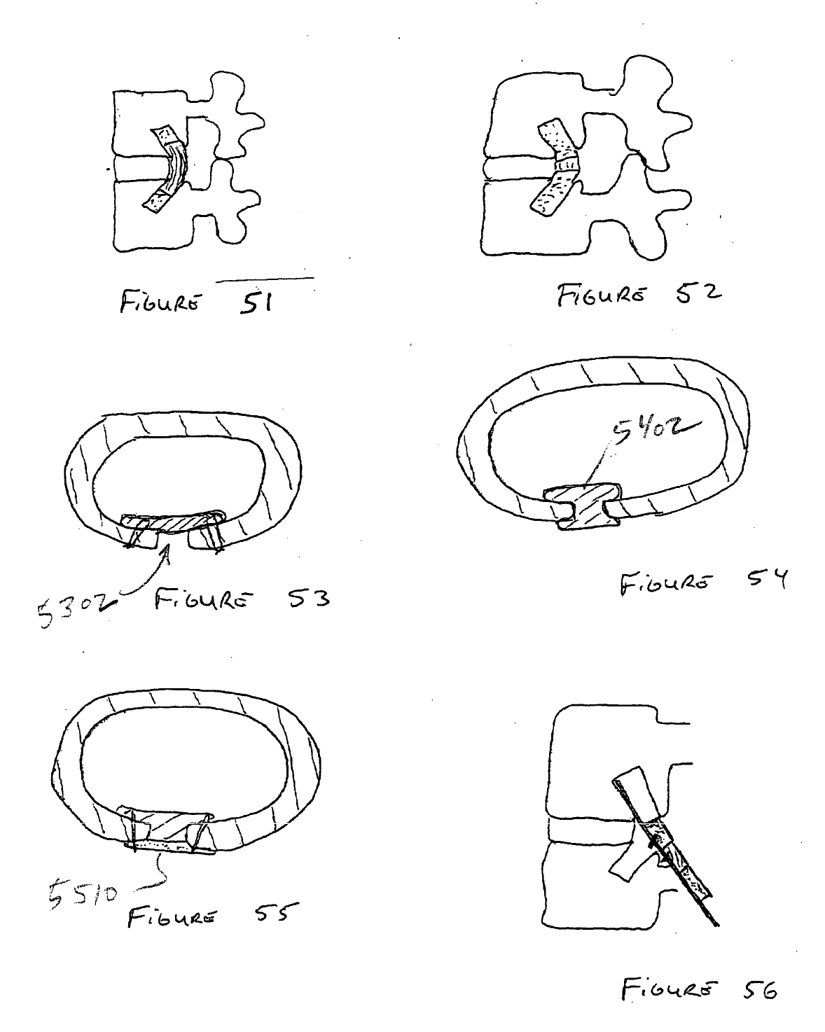

FIG. 54 is an axial cross section of a disc and an alternative embodiment of the invention drawn in FIG. 35B wherein the donor AF 5402 extends anterior and posterior to the defect in the host's AF. The graft is held in place by its attachment to the vertebrae. The press fit between the host AF and donor AF is insufficient to hold the graft within the AF. The invention relies on the superior fixation provided by attaching the graft to the vertebrae, not an optional press fit of the donor AF in the opening in the host's AF.

FIG. 55 is an axial cross section of a disc and an alternative embodiment of the invention drawn in FIG. 35B wherein sutures or other fixation devices pass through the host's AF, flaps from the donor AF, and a third component 5510. The third component lies on the spinal canal side of the host's AF. The third component may be a synthetic mesh as described in my U.S. Pat. 6,371,990, such as nylon. Alternatively, the third component may be made of allograft soft tissue.

FIG. 56 is a sagittal cross section of the spine and an alternative embodiment of the invention drawn in FIG. 46 wherein the loops of suture are passed over a K-wire. The tip of the K-wire has been placed into the hole in the vertebra. The graft is directed into the hole in the vertebra by pushing the graft along the K-wire. The K-wire is pulled out after placement of the graft. A cannulated interference screw may be placed before removal of the K-wire. A tool may be used to hold the graft, apply counter pressure, as the K-wire is extracted.

FIG. 57 is an axial cross section of disc, a novel Nucleus Replacement (NR), and an alternative embodiment of the invention drawn in FIG. 54. The three-part NR was inserted through two openings in the AF. The openings in the AF have been closed by two grafts 5702, 5704 using the embodiment drawn in FIG. 33. The invention is preferably used to augment the L5/S1 disc. The assembled NR is preferably between 5 to 20 mm tall, 15 to 40 mm wide, and 20 to 65 mm long. For example the assembled NR may be 12 mm tall, 25 mm wide and 45 mm long. The NR may be supplied in a kit that provides NRs and AR devices of different sizes.

FIG. 58 is an axial cross section of a disc, an alternative embodiment of the NR drawn in FIG. 57, and an alternative embodiment of the invention drawn in FIG. 33. The novel two-part NR was inserted through a single opening in the AF. The opening in the AF is preferably placed in an extraforaminal location. The invention is preferably used to augment the disc cephalad to the L5/S1 disc. The invention may also be used to augment the lumbar discs from a lateral approach and the cervical discs from an anterior approach.

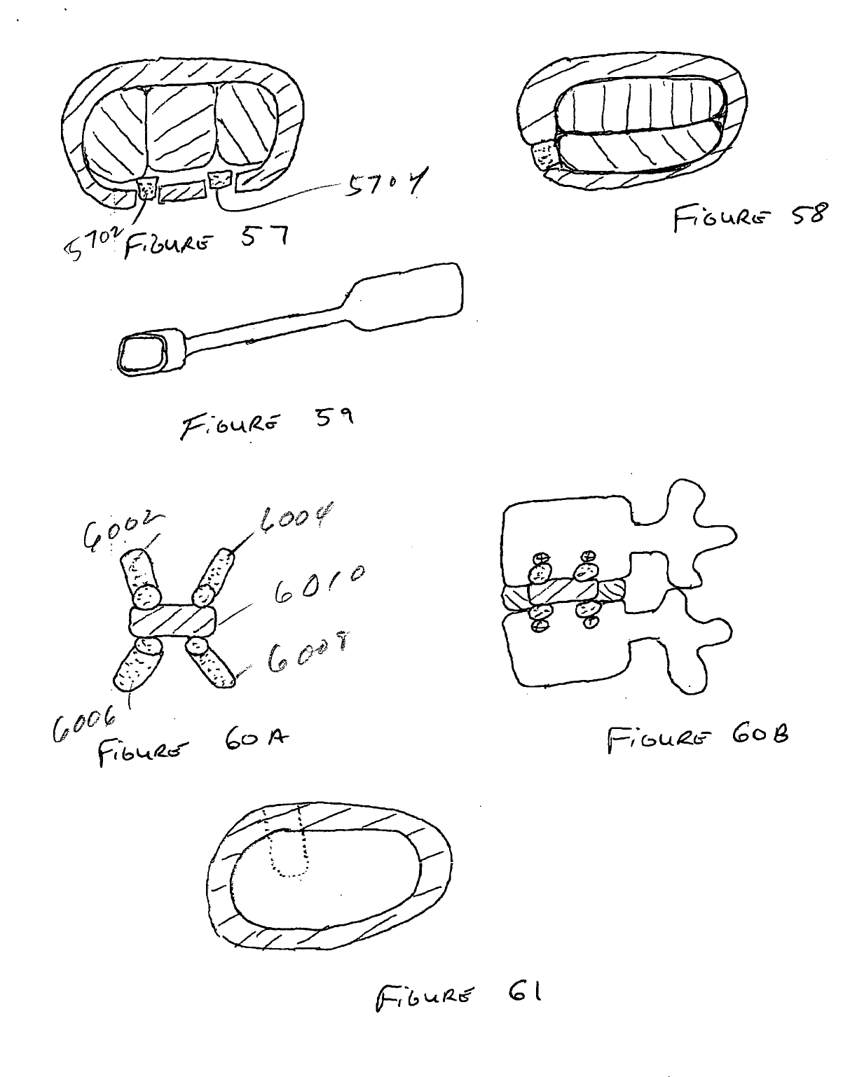

FIG. 59 is an oblique view of an instrument according to the invention which is used to create standard size openings in the AF. Tools with different sized cutting ends may be supplied to surgeons. Preferred cutting ends are 8×10 mm, 8×12 mm, 8×14 mm, 8×16 mm, and 8×18 mm. Alternative sizes and shaped cutting tools may be used. The tools preferably create rectangular or square openings with dimensions between 2 mm and 28 m. The cutting end of the tool is drawn on the left side of the drawing.

FIG. 60A is a lateral view of an alternative embodiment of the invention drawn in 13A. The graft has four bone components 6002, 6004, 6006, 6008 and a larger AF component 6010. The AF component may be 8-60 mm wide. FIG. 60B is a lateral view of the spine and the embodiment of the invention drawn in FIG. 60A. The invention may be used to close larger defects in the AF. The invention may also be used to replace or augment the entire posterior AF.

FIG. 61 is an axial cross section of a disc. The dotted line represents an alternative harvest site to that drawn in FIG. 21D. The harvest site includes a portion of the NP. My U.S. Pat. Nos. 6,340,359; 6,344,058; 6,352,557; 6,419,702 & 6,454,804 also teach transplantation of the NP and the NP with the AF.

FIG. 62 is a sagittal cross section of the spine and an alternative embodiment of the invention wherein the soft tissue device drawn in FIG. 16 is held in place by interference screws 6202, 6204 in holes drilled into the vertebrae.

FIG. 63 is a sagittal cross section of the spine and an alternative embodiment of the invention wherein a bone device 6302 is placed into a single vertebra. A portion of the bone device extends into the disc space. The invention may be used to prevent the extrusion intradiscal devices. The invention may also use of synthetic devices or synthetic devices that are filled with bone growth material. For example, the device could be made of titanium. Bone material or BMP impregnated collagen sponges (InFuse, Medtronic, Memphis Tenn.) could be packed into chambers with the device.

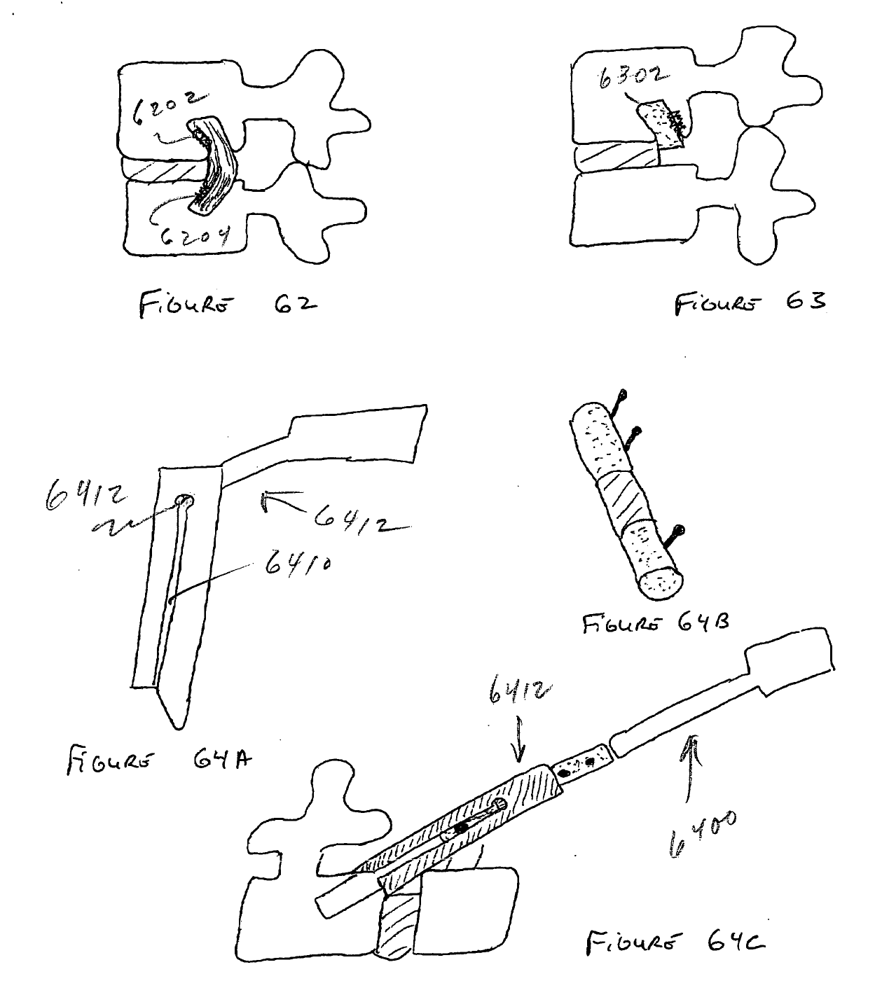

FIG. 64A is a lateral view of an alternative embodiment of the retractor drawn in FIG. 29A. The slot 6410 in the retractor is limited to a portion of the device 6412. The top of the slot has an enlarged opening 6412. FIG. 64B is an oblique view of an alternative embodiment of the invention drawn in FIG. 13A. Projections extend from the sides of the graft. The projections have enlarged tips.

FIG. 64C is a partial sagittal cross section of a portion of the spine and the embodiments of the invention drawn in FIGS. 64A and 64B. The projections from the graft pass through the slot in the retractor. The graft is directed into the hole in the vertebrae by cooperation between the projections and the slot in the sleeve. The drawing includes a pusher tool 6400.

FIG. 65 is a sagittal cross section of a portion of the spine and an alternative embodiment of the invention drawn in FIG. 25. One end of the graft is held by a screw 6502 that passes through at least a portion of the pedicle of the vertebra and the graft. Pins or other fixation devices may also be passed through the pedicle and the graft.

FIG. 66 is an anterior view of the spine and an alternative embodiment of the invention drawn in FIG. 25. The graft has been inserted into the anterior portion of the spine. This embodiment of the invention is preferably used in the cervical spine. The invention may be used to retain an intradiscal device.

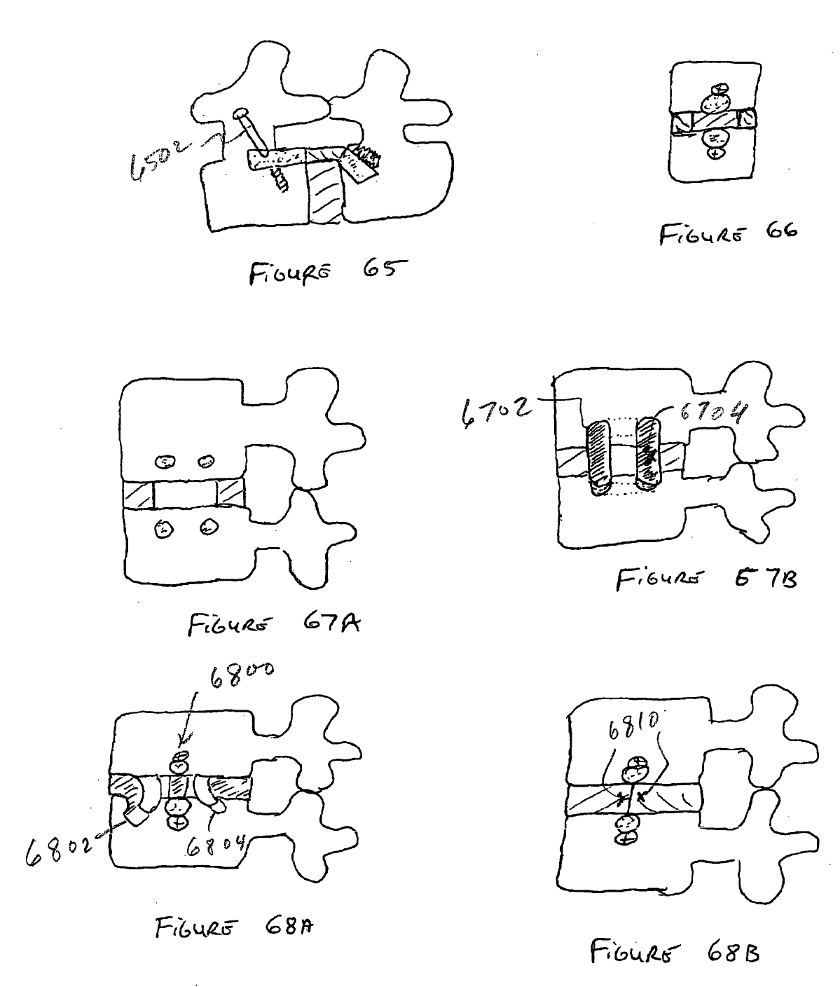

FIG. 67A is a lateral view of a portion of the spine and an alternative embodiment of the invention wherein an opening has been created in the lateral portion of the disc. A pair of holes can be seen above and below the disc. The holes in the vertebra above the disc connect with one another within the bone of the vertebra. Similarly, the holes in the vertebra below the disc connect with one another.

FIG. 67B is a lateral view of a portion of the spine and the embodiment of the invention drawn in FIG. 67A. A tendon 6702 has been passed through the holes in the vertebra. The ends of the tendon have been sewn together at 6704. The tendon lies over a portion of the opening in the AF. The embodiment of the invention may be used to retain intradiscal devices. Materials, other than tendon, may be used it the invention. For example, a titanium cable or a Gortex cord could be used in a similar fashion. Synthetic embodiments of the device may use alternative fastening mechanisms. For example, the ends of the synthetic embodiments could use the clasp mechanism commonly used in jewelry necklaces.

FIG. 68A is a lateral view of a portion of the spine and an alternative embodiment of the invention wherein flaps 6802, 6804 have been created in the AF. The embodiment of the invention drawn in FIG. 25 can be seen in the center of the defect in the AF. FIG. 68B is a lateral view of a portion of the spine and the embodiment of the invention drawn in FIG. 68A. The annular flaps have been sewn at 6810 to the novel graft 6800. The annular flaps may also be sewn to one another. The novel graft shields the reconstructed AF from pressure from intradiscal devices or intradiscal material.

FIG. 69 is an axial cross section of a disc and the embodiment of the invention drawn in FIG. 60A. The invention was used to reconstruct the entire posterior AF.

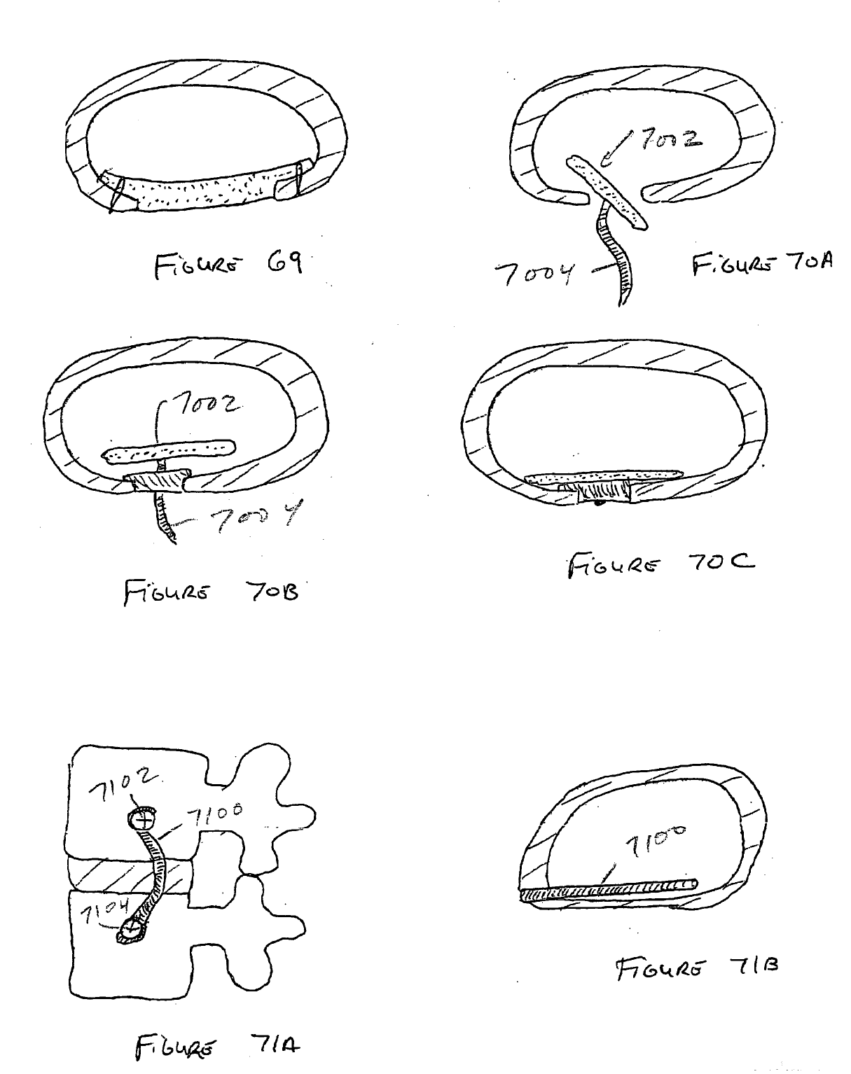

FIG. 70A is an axial cross section of the disc and an alternative embodiment of the invention. An intradiscal component 7002, as taught in my U.S. Pat. No. 6,245,107 and European patent EP 1180978 B1 and continuation-in-parts of the same including my U.S. patent application Ser. No. 10/185,284, is placed into the disc prior to placing the embodiment of the invention taught in FIG. 25. The intradiscal device may be made of natural or synthetic material. For example, the intradiscal component could be made of bone, cartilage, tendon, ligament, meniscus, titanium, plastic, PEEK, hydrogel, ceramic, nitinal, or other natural or synthetic material. A cord 7004 or cords is/are attached to the intradiscal component. The intradiscal component may be inserted at an angle to facilitate insertion of a stiff device through a small opening in the AF.

FIG. 70B is an axial cross section of the disc and the embodiment of the invention drawn in FIG. 70A. The invention taught in FIG. 25 has been inserted posterior to the intradiscal component. The suture, cord, or cable from the intradiscal component passes through the AF of the graft. FIG. 70C is an axial cross section of the disc and the embodiment of the invention drawn in FIG. 70B. The suture has been tightened. A crimp may be placed over the posterior portion of the suture. The suture holds the two components together. The intradiscal component helps protect the AF adjacent to the graft. The graft supports the intradiscal component. The intradiscal component applies little pressure to the host AF.

FIG. 71A is a lateral view of a portion of the spine and alternative embodiment of the invention taught in FIG. 1C. A sheet of allograft tissue 7100 is passed anterior to the posterior aspect of the AF. The allograft tissue is held in position be two screws 7102, 7104 that are placed across the vertebrae. The screws are preferably 3 to 10 mm in diameter and 15 to 60 mm in length. The screws are most preferably 8 mm in diameter and 50 mm in length.

FIG. 71B is an axial cross section of the disc and the embodiment of the invention taught in FIG. 71A. The device is preferably inserted from the left or right side of the spine. FIG. 71C is a coronal cross section of the spine and the embodiment of the invention drawn in FIG. 71A.

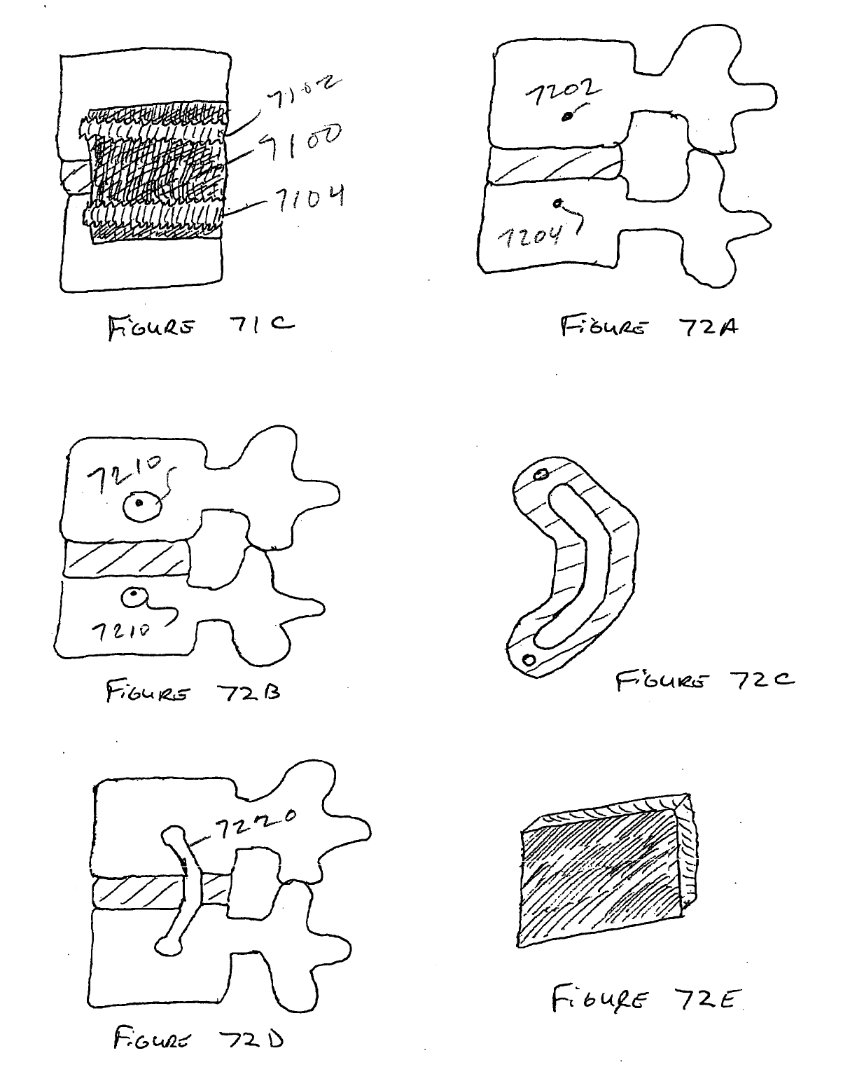

FIG. 72A is a lateral view of the spine and two K-wires 7202, 7204. The drawing illustrates the first step in inserting the embodiment of the invention taught in FIG. 71A. The K-wires are inserted across the vertebra above and below the disc. The position of the K-wires may be confirmed with intra-operative imaging such as fluoroscopy or CT scan. Positioning of the wires could be aided with navigational and/or robotic technology.

FIG. 72B is a lateral view of the spine and the embodiment of the invention drawn in FIG. 72A. The drawing illustrates use of a cannulated drill 7210 over the K-wires to enlarge the holes in the vertebrae. FIG. 72C is a lateral view of a guide that may be used with the embodiment of the invention drawn in FIG. 72B. The guide may be passed over the K-wires. The guide wires may be passed through the holes at the ends of the guide. A saw or burr may be passed through the slot in the guide to create a slot in the vertebrae and the disc.

FIG. 72D is a lateral view of a portion of the spine. A slot 7220 has been created in the lateral portion of the spine using the invention taught in FIGS. 72A to 72C. Allograft tissue, such as fascia, is placed in the slot and across the vertebrae. The screws are placed after insertion of the soft tissue graft. FIG. 72E is an oblique view of the tissue used in the embodiment of the invention drawn in FIG. 71A.

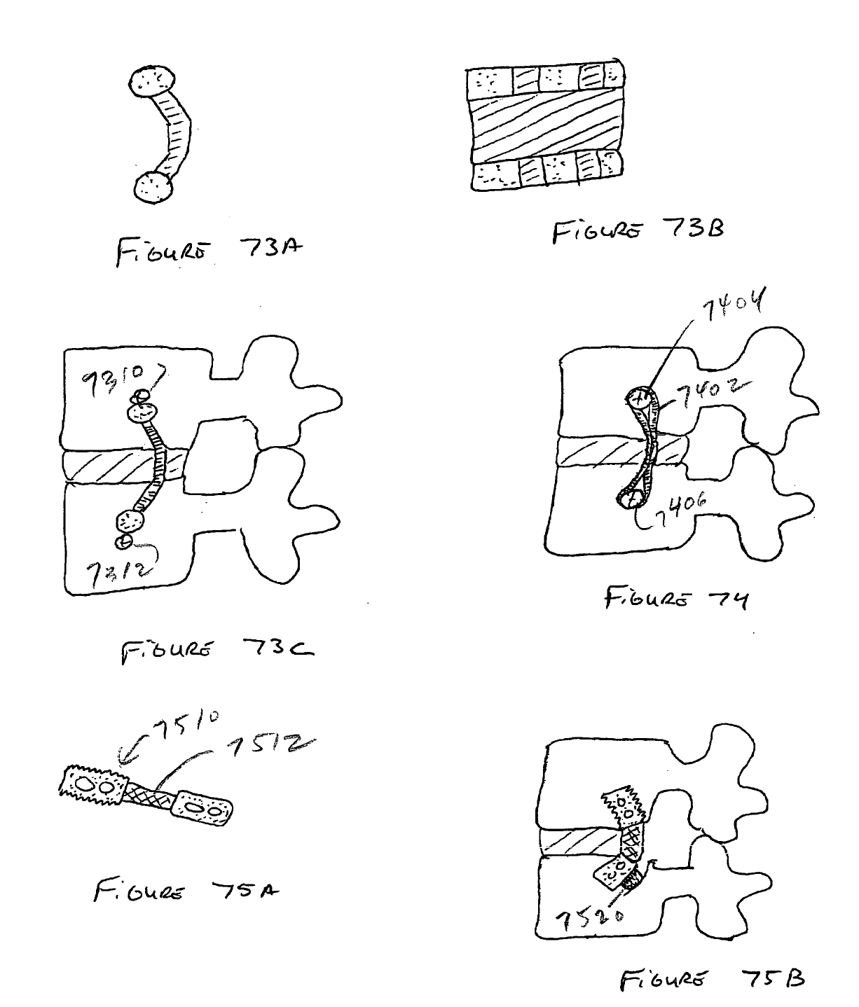

FIG. 73A is a lateral view of an alternative embodiment of the invention drawn in FIG. 72E. The ends of the device are thicker than the central portion of the device. The device may be manufactured of allograft bone (dotted area of the drawing) and allograft soft tissue (area of the drawing with horizontal lines). Alternatively, the dowel-shaped ends of the device could be made of a synthetic material that promotes bone ingrowth. For example, the ends of the device could be made of tantalum, titanium, ceramic, or other material. The surface of the ingrowth material could be treated to promote bone ingrowth. For example, the ingrowth surfaces could be covered with plasma spray, metal beads, or collagen soaked BMP sponges to promote bone healing. The flexible portion of the device could be made for synthetic materials such as nylon or Gortex. The flexible component could be covered with material that promotes soft tissue ingrowth. For example, a nylon mesh component could be covered with allograft tissue, collagen, or intestinal submucosa. Alternatively, the flexible component could be made entirely of allograft soft tissue such as fascia, tendon, or ligament. The bone ingrowth components are preferably 2 to 20 mm in diameter and 10 to 60 mm in length. The bone ingrowth components are most preferably 8 mm in diameter and 45 mm in length. The flexible component between the bone ingrowth components is preferably 2 to 10 mm in thickness, as wide as the bone ingrowth components, and 10 to 70 mm in height. The flexible component is most preferably 3 mm thick and 45 mm in height.

FIG. 73B is an anterior view of the embodiment of the device drawn in FIG. 73A. Straps of the flexible component surround recessed areas of the bone ingrowth component. Alternative fastening mechanisms may be used. For example, the flexible component may be molded around the bone ingrowth component, molded into slots of the bone ingrowth components, or crimped into slots in the bone ingrowth components.

FIG. 73C is lateral view of the spine and the embodiment of the invention drawn in FIG. 73B. The device is held in place with interference screws 7310, 7312. Alternatively, the device may be held in place with staples or a plate and screws. The AF may be reinforced by attaching synthetic mesh to the lateral surface of the AF on either side of the flexible component. The synthetic mesh component would preferably be 1 to 2 mm thick, 10 to 30 mm long and 8 to 30 mm tall. A portion of the mesh may be attached to the vertebra above and/or below the disc.

FIG. 74 is a lateral view of the spine and an alternative embodiment of the invention wherein flexible component 7402 is looped around two screws 7404, 7406. The flexible component may be made of the natural or synthetic materials described in the text of the FIGS. 71A to 73C.

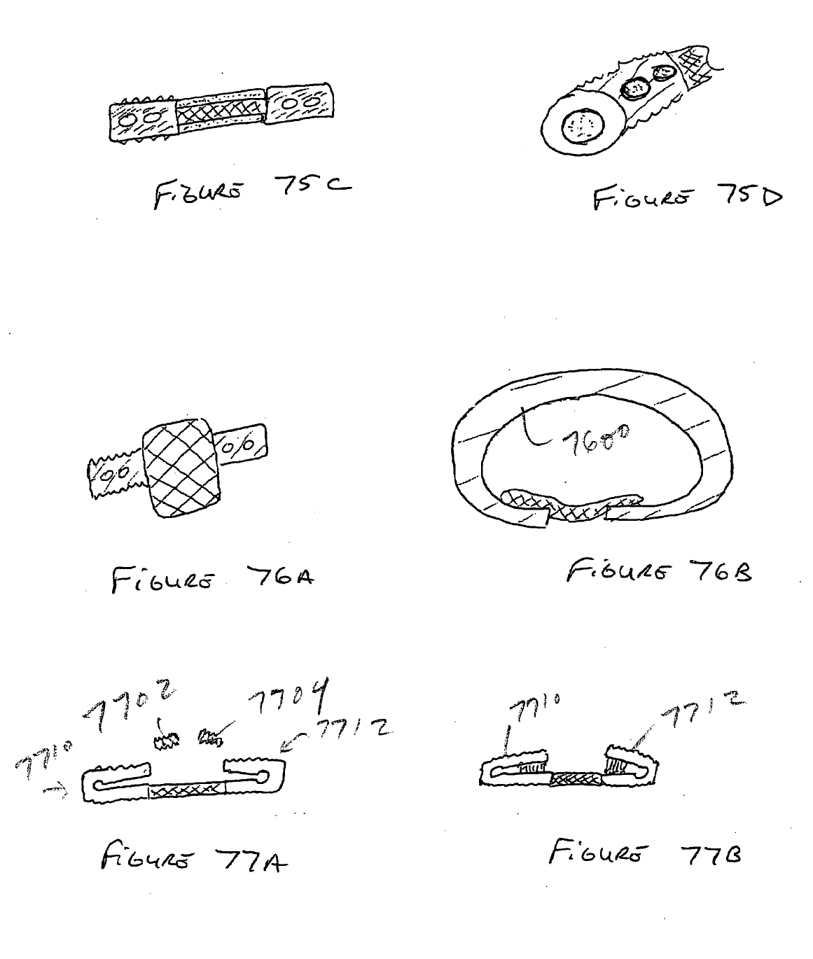

FIG. 75A is a lateral view of an alternative embodiment of the invention drawn in FIG. 13A. The device includes components made of synthetic materials. The bone ingrowth components at the ends of the device may be made of metals such as titanium, tantalum, chrome cobalt, ceramic, PEEK (polyaryletherketone), Polyphenolsulfone, Polysulfone, Acetal (Delrin), UHMW Polyethylene, and composites of these materials and carbon fibers or resorbable materials listed in the text of other embodiments of the invention. The surfaces of the bone ingrowth components may be treated to promote bone ingrowth. The bone ingrowth components may be covered with plasma spray, beads, or hydroxyappetite. The bone ingrowth components may contain chambers. The chambers may be filled with bone or material that promotes bone ingrowth including demineralized bone, BMP soaked collagen sponges (most preferably BMP 2 or BMP 7, but may include BMP 2 to BMP 14 to BMP n). The bone ingrowth components may also be wrapped with BMP soaked collagen sponges.

One of the bone ingrowth components 7510 is preferably threaded. The flexible component 7512 between the bone ingrowth components is preferably made of at least one synthetic material. The flexible component is preferably made of materials that are flexible but than have high tensile strength. Materials such as nylon or Gortex mesh may be used. The flexible member preferably promotes tissue ingrowth. Synthetic mesh could be covered impregnated with collagen, intestinal submucosa, allograft tissue, or BMP soaked collagen sponges. Stem cells may be added to the bone ingrowth components and/or the flexible component. Stem cells may be harvested by forcing an aspirate from the patient's bone marrow through the Cell Select System supplied by DePuy in Raynham, Mass. The synthetic embodiment of the invention would be supplied in the sizes listed in the text of FIG. 13A. The synthetic and allograft devices of various sizes are preferably included in a kit. The kit includes instruments of sizes that facilitate insertion of AR devices of various sizes.

FIG. 75B is a lateral view of the spine, the embodiment of the invention drawn in FIG. 75A, and an alternative embodiment of the invention drawn in FIG. 25. The first end of the device is threaded into the vertebra above the disc. The device may be threaded into a pilot hole in the vertebra. Alternatively the device may be threaded over a K-wire that is placed in the vertebra above the disc. The second end of the device is held in the vertebra below the disc by an interference screw 7520. Alternative fixation methods are acceptable including cross pins, Polymethylmethacrylate, or other fixation techniques well know to Orthopaedic or Neurosurgeons.

FIG. 75C is a lateral view of an alternative embodiment of the invention drawn in FIG. 75A. The synthetic mesh of the flexible component has been covered or filled with a material that promotes tissue ingrowth. FIG. 75D is an oblique view of a portion of the embodiment of the invention drawn in FIG. 75A. The bone ingrowth component has been filled with a material that promotes bone ingrowth.

FIG. 76A is a lateral view of an alternative embodiment of the invention drawn in FIG. 75A. The flexible component is wider than the bone ingrowth components. FIG. 76B is an axial cross section of a disc and the embodiment of the invention drawn in FIG. 76A. The flexible component extends inside the AF 7600. Alternatively, the flexible component could extend outside the AF or both inside and outside the AF. As mentioned in the other embodiments of the invention, the flexible component could be fastened to the AF with sutures, staples, biocompatible glue or other mechanisms.

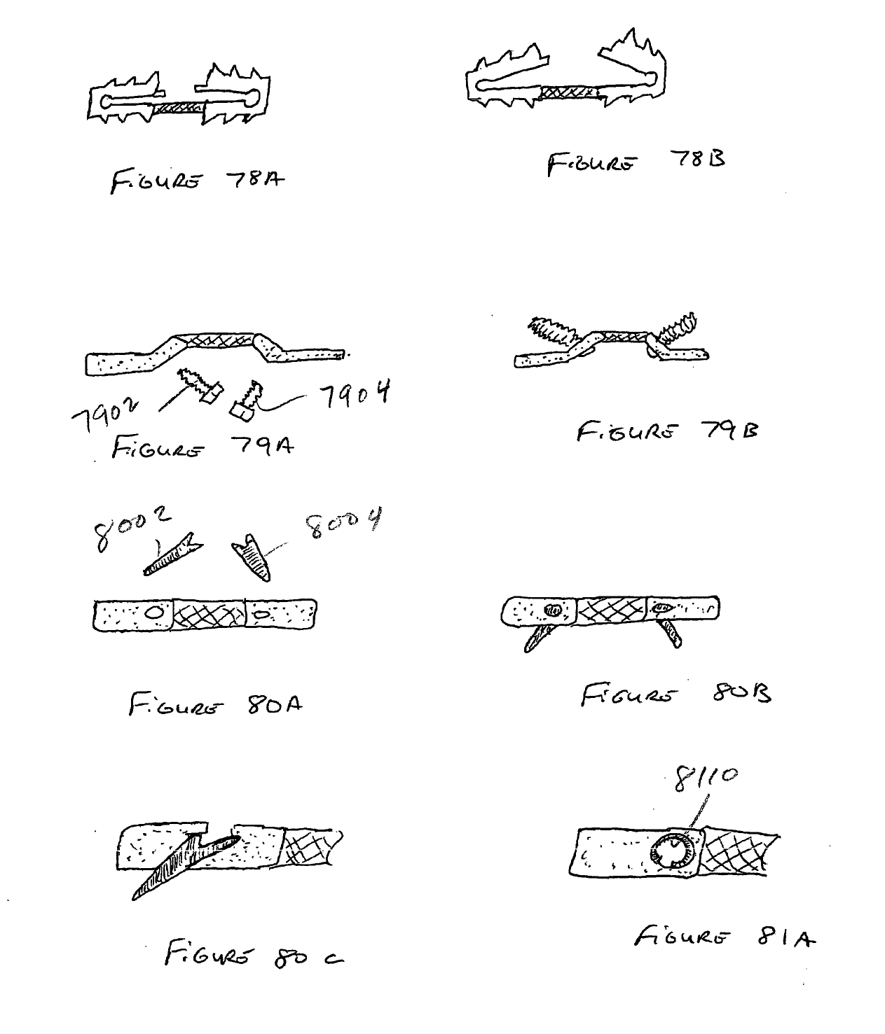

FIG. 77A is an exploded lateral view of an alternative embodiment of the invention drawn in FIG. 75A. Screws 7702, 7704 may be threaded into the bone ingrowth components 7710, 7712 to expand the bone ingrowth components after placing the device in the spine. FIG. 77B is a lateral view of the embodiment of the invention drawn in FIG. 77A. The screws have been threaded into the bone ingrowth components, thus expanding the bone ingrowth components. Other mechanical expansion technology can be used in other embodiments of the invention. For example, threaded wedges may be used to expand the bone ingrowth components or technology used in expanding drywall “Molly” fasteners may be used.

FIG. 78A is a lateral view of an alternative embodiment of the invention wherein the bone ingrowth components are made of materials with elasticity or shape memory. The bone ingrowth components expand after placement in the spine without secondary steps. For example, a spring-biased component could be released, thus allowing the device to expand, after placement in the spine. Components made of shape memory materials such as Nitinol, could expand within the spine in reaction to a temperature, chemical, or other change or stimulus. A tool could clamp the projections near the flexible member to compress elastic bone ingrowth components. FIG. 78B is a lateral view of the embodiment of the invention drawn in FIG. 78A. The device is drawn in its expanded configuration.

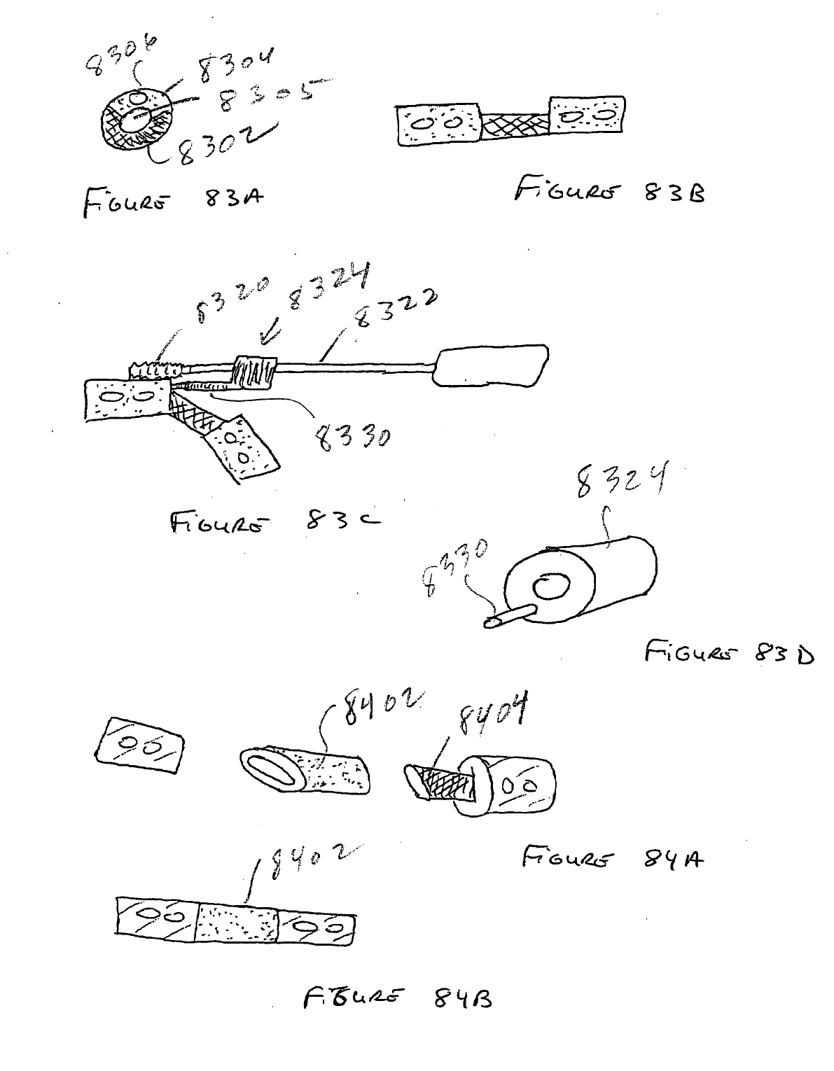

FIG. 79A is an exploded lateral view of an alternative embodiment of the invention drawn in FIG. 78A. The screws 7902, 7904 are preferably placed through holes near the junction of the bone-ingrowth members and the flexible component. FIG. 79B is a lateral view of the assembled embodiment of the invention drawn in FIG. 79A. The screws diverge from the long axis of the bone ingrowth components. Diverging components increase the device's resistance to migration. The screws may be threaded into the bone ingrowth component. Anti-back out mechanisms, as used in cervical plate technology, may be used to capture the screws in the bone ingrowth component. FIGS. 81A-81C illustrates one such capture mechanism.

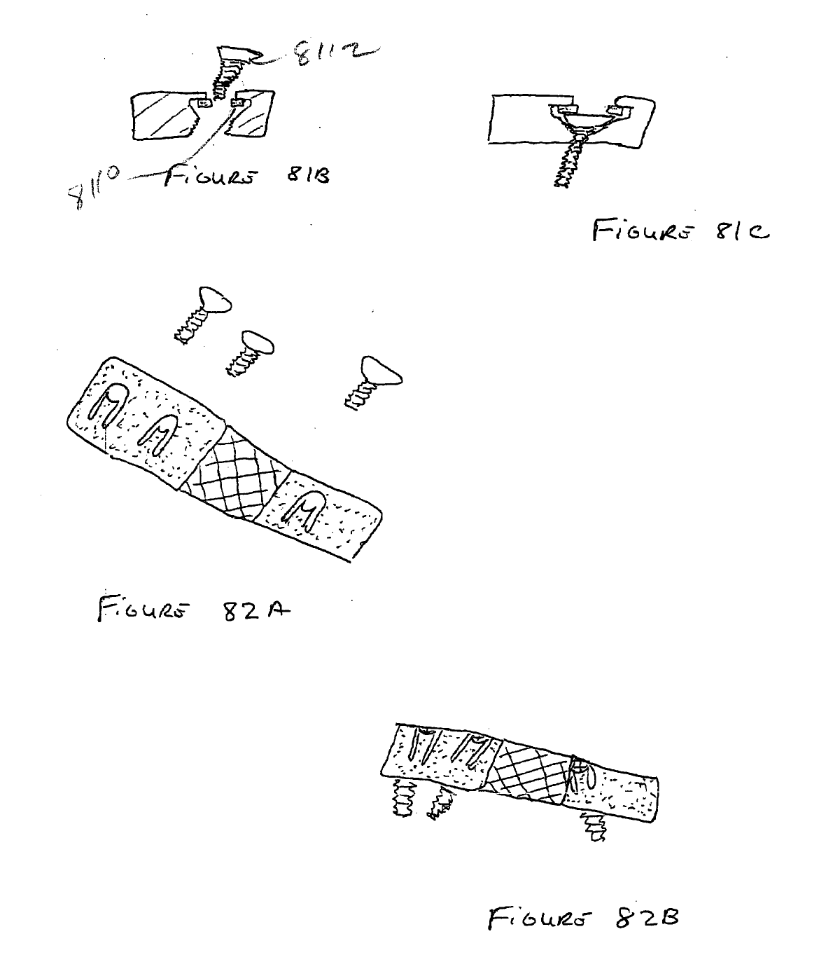

FIG. 80A is an exploded lateral view of an alternative embodiment of the invention drawn in FIG. 79A. FIG. 80B is a lateral view of the assembled embodiment of the invention drawn in FIG. 80A. Pins 8002, 8004 are placed through holes in the bone ingrowth components. The pins diverge from the long axis of the bone ingrowth components. The pins are added to the device after the device is placed in the spine.

FIG. 80C is a sagittal cross section of a portion of the embodiment of the invention drawn in FIG. 80B. The pins are captured by the bone ingrowth component. The tips of the pin expand, after the pin is forced below the overhanging portions of the bone-ingrowth component. Alternative capture mechanisms including shape memory technology may be used in alternative embodiments of the invention.

FIG. 81A is the view of the top of a portion of the embodiment of the invention drawn in FIG. 79A. The bone-ingrowth component contains an expandable C-ring 8110. FIG. 81B is sagittal cross section of the embodiment of the invention drawn in FIG. 81A. The screw 8112 wedges the C-ring open as the head of the screw passes through the C-ring. The hole in the bone ingrowth component may be threaded to help drive the screw through the C-ring.

FIG. 81C is a sagittal cross section of the embodiment of the invention drawn in FIG. 81B. The screw has been passed below the C-ring. The screw is captured by the C-ring and the bone-ingrowth component.

FIG. 82A is an exploded lateral view of an alternative embodiment of the invention drawn in FIG. 80A. The bone-ingrowth components are configured to expand as the heads of the screws pass through. The elasticity of the material returns the ingrowth components to their original shape after placement of the screws. The holes in the bone ingrowth components contract as the components return to their original shape, thus capturing the screws in the device. FIG. 82B is a lateral view of an assembled embodiment of the invention drawn in FIG. 82A.

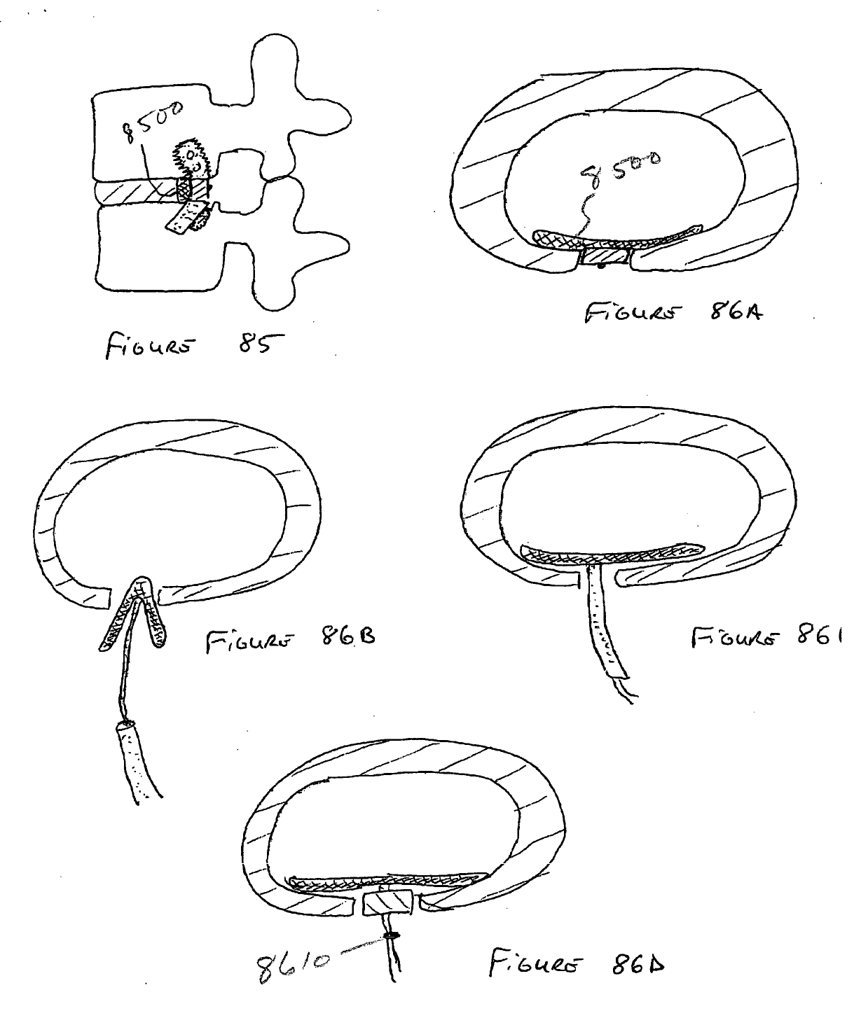

FIG. 83A is an axial cross section through the flexible component of an alternative embodiment of the invention drawn in FIG. 75A. The cross-hatched area 8302 on the bottom of the drawing represents the cross section of the flexible component. The bone-ingrowth component 8304 contains a larger hole 8305 to hold bone-ingrowth material and a smaller hole 8306 on the top of the device. The smaller hole cooperates with a projection from a tool that guides interference screws along the bone ingrowth component.

FIG. 83B is a lateral view of the embodiment of the invention drawn in FIG. 83A. FIG. 83C is a lateral view of the embodiment of the invention drawn in FIG. 83B, an interference screw 8320, a screwdriver 8322, and a novel guide 8324. The projection 8330 from the guide fits into the hole in the bone-ingrowth component. The close fit between the guide and the shaft of thee screwdriver, as well as the cooperation between the guide and the bone-ingrowth component, direct the screw along the bone-ingrowth component. FIG. 83D is an oblique view of the tip of the guide drawn in FIG. 83C.

FIG. 84A is an exploded lateral view of an alternative embodiment of the invention drawn in FIG. 75C. A sleeve of material 8402 that promotes soft tissue ingrowth is placed over the flexible high tensile strength component 8404 during the manufacturing process. The device can be manufactured with other techniques. For example a sheet of tissue ingrowth material could be wrapped around the high tensile strength component and fastened to the high tensile strength component. Alternatively, the tissue ingrowth material could be at least partially cured over the high tensile strength component. Curing materials include polyurethane, compounds or solutions of collagen, or other materials. FIG. 84B is lateral view of the assembled embodiment of the invention drawn in FIG. 84A.

FIG. 85 is a sagittal cross section of a portion of the spine and an alternative embodiment of the inventions drawn in FIGS. 75C and 70C. An intradiscal dam component 8500 is attached to the anterior surface of the flexible component of the device drawn in FIG. 75C.

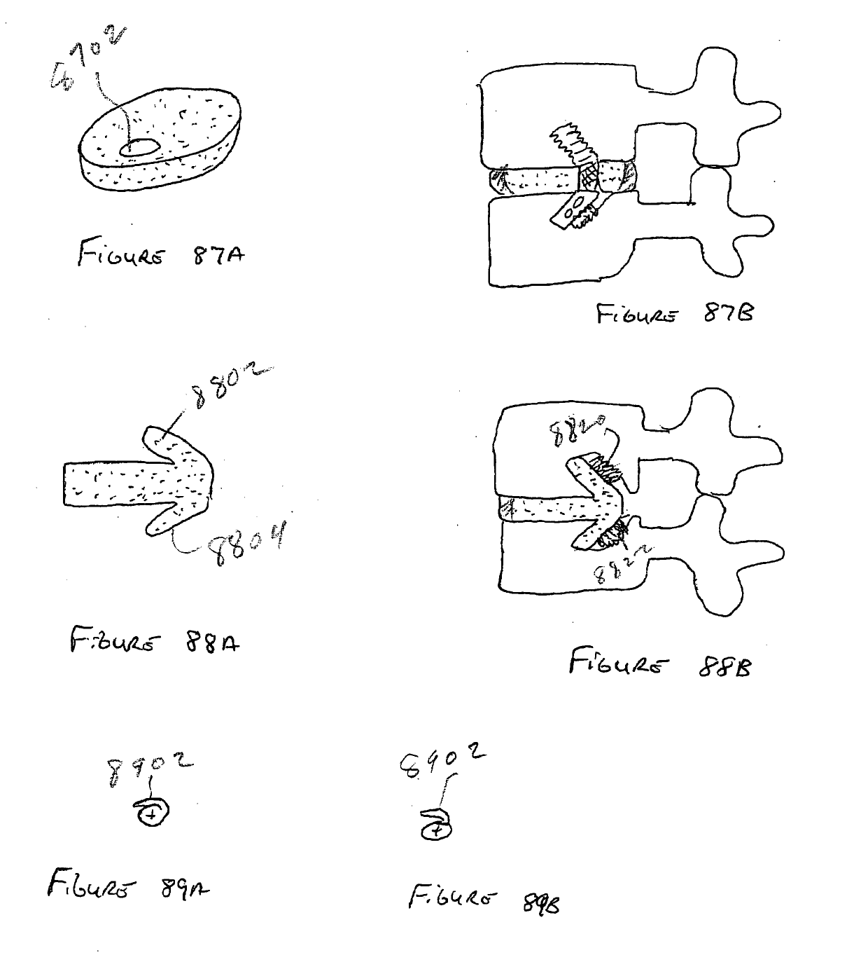

FIG. 86A is an axial cross section of a disc and the embodiment of the invention drawn in FIG. 85. The intradiscal dam component 8500 extends inside the patient's AF. The intradiscal component is fastened to the device drawn in FIG. 75C. As described in the text of other embodiments of the invention, the intradiscal component may also be fastened to the patient's AF. FIG. 86B is an-axial cross section of a disc and the first step in the insertion of the intradiscal component drawn in FIG. 86A. The intradiscal component is shaped to facilitate insertion of the component through a small opening in the AF.

FIG. 86C is an axial cross section of a disc and the embodiment of the invention drawn in FIG. 86B. The intradiscal component is drawn in a second shape. The component has expanded in a left to right direction to extend over the patient's AF. The component may expand by its elasticity, as a tool is removed thus releasing compression of the component. Alternatively the component may be made of a shape memory material such as Nitinol that expands, or changes shape as a reaction to temperature change or other stimulus. A hydrogel intradiscal component could expand as it absorbs fluids. Alternative embodiments of the intradiscal component include components made of in-situ curing materials such as polyurethane or a collagen containing solution or compound.

FIG. 86D is an axial cross section of a disc and the embodiment of the invention drawn in FIG. 86C. The intradiscal component is fastened to the embodiment of the invention drawn in FIG. 75C. Alternatively, the intradiscal component may lie against but not be fastened to the embodiment of the invention drawn in FIG. 75C. The embodiment of the invention drawn in FIG. 75C supports the intradiscal component. The embodiment of the invention drawn in FIG. 75C prevents the intradiscal component from deforming enough to allow the intradiscal component to extrude through the opening in the AF in reaction to large forces placed on the intradiscal component by the patient's NP or other intradiscal device. The drawing also illustrates the use of a crimp 8610 to tether the suture or cord extending from the intradiscal component. Excessive suture may be cut from the device after the crimp is closed over the suture.

FIG. 87A is an oblique view of a novel intradiscal device such as a nucleus replacement which has a hole 8702. The intradiscal device is preferably made of compressible polymers such as polyurethane, silicon, or hydrogel. FIG. 87B is a sagittal cross section of a portion of the spine, the embodiments of the invention drawn in FIG. 75C and 87A. The flexible component of the device drawn in FIG. 75C passes through the hole in the device drawn in FIG. 87A. The embodiment of the device drawn in FIG. 75C prevents the intradiscal device from extruding through the AF or from applying too much pressure to the AF. The embodiment of the invention drawn in FIG. 35A also decreases the force intradiscal devices may apply to the AF by extending anterior to the AF.

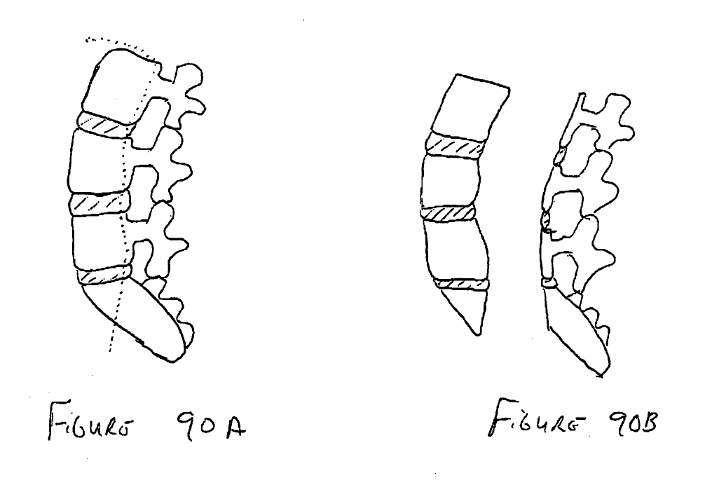

FIG. 88A is a lateral view of an alternative embodiment of the invention drawn in FIG. 87A. FIG. 88B is a sagittal view of a portion of the spine and the embodiment of the invention drawn in FIG. 88A. Projections 8802, 8804 from the intradiscal device 8810 extend into holes drilled in the vertebra above and/or below the disc. The intradiscal device is held in place with interference screws 8820, 8822. The technique to drill the holes, insert the screws, or other fastening methods are described in the text of other embodiments of the invention.

FIG. 89A is a view of the head of a novel interference screw according to the invention which has a projection 8902 that prevents anti-rotation and interference screw backout. FIG. 89B is a view of the head of the embodiment of the interference screw drawn in FIG. 89A. The projection is drawn in its extended position. The projection extends as the screw is rotated in the opposite direction of its insertion.

FIG. 90A is a lateral view of a portion of the spine. The dotted line represents a method of harvesting portions of a donor spine, without cutting the spinal cord, thecal sack, or the spinal nerves. The vertebrae and the discs are transected anterior to the spinal canal. The muscles and spinal nerves are elevated from the anterior and lateral portions of the spine prior to cutting the spine. The cuts could be made with a power saw, power burs, and/or a gigli saw. The invention may be used to reduce the risk of disease transmission from donors who have infectious diseases in their neural tissues. FIG. 90B is an exploded lateral view of the spine in the embodiment of the invention drawn in FIG. 90A.

The methods and apparatus taught herein prevent excessive pressure on the AF. In the preferred embodiment, a nucleus replacement (NR) is inserted from a lateral aspect of the lumbar spine at L4/L5 or above. A transpsoas technique is used in the preferred lateral approach to the lumbar spine. Identification of nerves within the psoas muscle allows the nerves to be protected and retracted anterior and posterior as necessary. A nerve stimulator may be used to help identify the nerves. Alternatively, the psoas muscle could be split with an endoscope that delivers or records electrical impulses. The myotomes of the extremities could be monitored to detect the electrical impulses. The novel NR could also be used from an anterior approach to the cervical spine. The dam or barrier embodiments of the invention may be used in conjunction with natural as well as artificial discs.

1. A spinal repair system that alleviates pressure on the annulus fibrosis, nerves, or other delicate body structures, comprising:
a first end portion adapted for placement within an intervertebral body;
a second end portion adapted for placement within an adjacent intervertebral body; and
a bridge portion connecting the first and second end portions, the bridge portion being adapted to span a portion of an intervertebral disc space and prevent excessive outward bulging.

2. The system of claim 1, wherein the first and second end portions are composed of a rigid biocompatible material.

3. The system of claim 1, wherein the first and second end portions are composed of allograft bone.

4. The system of claim 1, wherein the bridge portion is composed of a flexible, braided or mesh material.

5. The system of claim 1, wherein the bridge portion is composed of allograft annulus fibrosis.

6. The system of claim 1, wherein:
the first and second end portions are composed of allograft bone; and
the bridge portion is composed of allograft annulus fibrosis.

7. The system of claim 1, further including screws to hold the first and second end portions into respective vertebral bodies.

8. The system of claim 1, wherein:
the first and second end portions are elongate; and
the bridge portion spans the end portions in a plane parallel to the end portions.

9. The system of claim 1, wherein:
the first and second end portions are elongate cylinders;
the system further comprising slotted bone dowels into which the end portions are received; and
the bridge portion extends through one slot and into the other when implanted.

10. The system of claim 1, further including:
an artificial disc replacement (ADR) defining a volume; and
wherein the bridge portion extends through at least a portion of the volume of the ADR.

11. The system of claim 1, wherein the end and bridge portions together form a cylindrical shape.

12. The system of claim 1, wherein at least one of the end portions is threaded.

13. The system of claim 1, wherein one or both of the end portions are configured for bony ingrowth.

14. A spinal repair method, comprising the steps of:
providing the system of claim 1;
forming a first hole or channel in a first intervertebral body;
placing the first end portion into the first hole or channel;
forming a second hole or channel in an adjacent intervertebral body;
placing the second end portion into the second hole or channel such that the bridge portion spans a hole or defect in an annulus fibrosis.

15. The method of claim 14, including the step of securing the end portions with screws.

16. The method of claim 14, wherein the step of providing the system of claim 1 includes harvesting the portions from a human or animal donor, with the end portions comprising intervertebral bone and the bridge portion comprises annulus fibrosis still attached to the end portions.
